SEQUENCE LISTING (12 sequences)

SEQ ID NO: 1 - protein, 5 aa, Artificial Sequence
  RGD sequence from  fibronection
YIGSR

SEQ ID NO: 2 - protein, 34 aa, Homo sapiens
SVSEIQLMHNLGKHLNSMERVEWLRKKLQDVHNF

SEQ ID NO: 3 - protein, 5 aa, Artificial Sequence
  Synthetic plasmin degradable sequence
GYKNR

SEQ ID NO: 4 - protein, 11 aa, Artificial Seqence
  Synthetic adhesion peptide
GCGYGRGDSPG

SEQ ID NO: 5 - protein, 16 aa, Artificial Sequence
  Synthetic MMP substrate
GCRDGPQGIAGFDRCG

SEQ ID NO: 6 - protein, 16 aa, Artificial Sequence
  Synthetic crosslinker peptide
GCRDGPQGIWGQDRCG

SEQ ID NO: 7 - protein, 23 aa, Artificial Sequence
  Synthetic peptide
GKGKGPQGIAGQKGPQGIAGQKG

SEQ ID NO: 8 - protein, 21 aa, Artificial Sequence
  Synthetic peptide
GKGPQGIAGQKGPQGIAGQKG

SEQ ID NO: 9 - protein, 16 aa, Artificial Sequence
  Synthetic peptide
GCRDGPQGIWGQDRCG

SEQ ID NO: 10 - protein, 16 aa, Homo sapiens
  PEPTIDE (1)..(16): Peptide found in natural collagen type II alpha chain
GCRDGPQGIAGQDRCG

SEQ ID NO: 11 - protein, 16 aa, Artificial Sequence
  Synthetic peptide
GCRDGPQGIWGQDRCG

SEQ ID NO: 12 - protein, 16 aa, Artificial Sequence
  Synthetic peptide
GCRDGPQGIAGQDRCG

(19) United States
(12) Patent Application Publication
(10) Pub. No.: US 2014/0343324 A1
(43) Pub. Date: Nov. 20, 2014

(54) SYNTHETIC MATRIX FOR CONTROLLED CELL INGROWTH AND TISSUE REGENERATION

(71) Applicants: Eidgenossische Technische Hochschule Zürich, Zurich (CH); Universität Zürich, Zurich (CH)

(72) Inventors: Matthias Lütolf, Zurich (CH); Jason C. Schense, Zurich (CH); Anna Jen, Zurich (CH); Marina Capone, Zurich (CH); Jeffrey A. Hubbell, Lausanne (CH)

(73) Assignees: Eidgenossische Technische Hochschule Zürich, Zurich (CH); Universität Zürich, Zurich (CH)

(21) Appl. No.: 14/283,837

(22) Filed: May 21, 2014

Related U.S. Application Data
(63) Continuation of application No. 11/735,263, filed on Apr. 13, 2007, now abandoned.
Application No. 11/735,263 is a continuation-in-part of application No. 10/494,905, filed on Oct. 25, 2004, now abandoned.
(60) Provisional application No. 60/337,783, filed on Nov. 7, 2001.

(51) Int. Cl. C07C 317/04

(52) U.S. Cl. CPC C07C 317/04
USPC 568/32

(57) ABSTRACT

Biomaterials containing a three-dimensional polymeric network formed from the reaction of a composition containing at least a first synthetic precursor molecule having n nucleophilic groups and a second precursor molecule having m electrophilic groups wherein the sum of n+m is at least five and wherein the sum of the weights of the first and second precursor molecules is in a range from about 8 to about 16% b weight of the composition, preferably from about 10 to about 15%, more preferably from about 12 to about 14.5% by weight of the composition. In one embodiment, the first and second precursor molecules are polyethylene glycols functionalized with nucleophilic and electrophilic groups, respectively. In a preferred embodiment, the nucleophilic groups are amino and/or thiol groups and the electrophilic groups are conjugated, unsaturated groups. The ratio of the equivalent weights of the electrophilic groups (second precursor molecule) and the nucleophilic groups (first precursor molecule) is in the range of between 0.7 and 1.1, more preferably between 0.8 and 1.0. The first and/or second precursor molecule may be covalently bound to one or more molecules selected from the group consisting of cell adhesion peptides, growth factors, and growth factor-like peptides.

### CROSS-REFERENCE TO RELATED APPLICATIONS

This application is continuation-in-part of U.S. Ser. No. 10/494,905, filed May 7, 2004, which is a 371 of PCT/EP02/12458, filed Nov. 7, 2002, which claims priority to U.S. Ser. No. 60/337,783 filed Nov. 7, 2001.

### FIELD OF THE INVENTION

The present invention is in the field of polymeric matrices, particularly synthetic polymeric matrices, for wound healing applications and tissue regeneration.

### BACKGROUND OF THE INVENTION

The use of biomaterials as three dimensional scaffolds or matrices (with or without bioactive factors attached) for wound healing applications and tissue regeneration has been described in the literature. For application in the body, in-situ formation of the matrix at a particular site in the body is often preferable over implantation of preformed biomaterials which require invasive surgery, can be difficult to sterilize, and often do not match the shape of the defect. Moreover, the requirement of biocompatibility limits the choice of chemistry both with regard to the nature of the precursor molecules as well as the crosslinking chemistry used for the in-situ formation of the matrix.

Various precursor molecules able to form matrices at a desired site in the body have been described. Naturally occurring materials, synthetic materials, semi-synthetic materials, and combinations thereof have been used. Matrices based on naturally occurring or chemically modified naturally occurring proteins, such as collagen, denatured collagen (gelatin), and in particular fibrin, have been used with some success. Other examples include carbohydrates, like cellulose, alginates and hyaluronic acid.

Naturally occurring materials, however, can suffer from a variety of limitations such as immunogenicity, costly production methods, limited availability, batch variability and difficulty purifying the materials. These problems can limit the use of matrices formed from naturally occurring precursors.

In an attempt to overcome the problems associated with naturally occurring materials, matrices based on synthetic precursor molecules have been developed for tissue regeneration in and/or on the body. The crosslinking reactions used for the formation of the synthetic matrices in the body include (i) free-radical polymerization between two or more precursors containing double bonds, as described in Hubbell et al., J. Biomed. Mater. Res. 39:266-276, 1998, (ii) nucleophilic substitution reactions e.g., between a precursor comprising a nucleophilic group such as an amine group and a precursor comprising an electrophilic group, such as a succinimidyl group as disclosed in U.S. Pat. No. 5,874,500 to Rhee et al., (iii) condensation and addition reactions and (iv) Michael type addition reaction between a strong nucleophile (e.g., thiol or amino groups) and a strong electrophile (e.g., conjugated unsaturated groups, such as acrylate or vinyl sulfone groups). Michael type addition reactions are described in WO00/44808, the content of which is incorporated herein by reference.

Michael type addition reactions allow for in situ crosslinking of at least a first and a second precursor molecule under physiological conditions in a self-selective manner. Thus, even in the presence of reactive biological materials, the precursor molecules react much faster with each other to form the matrix than they react with other molecules in the biological environment. When one of the precursor molecules has a functionality of at least two, and at least one of the other precursor molecules has a functionality greater than two, the system will self-selectively react to form a cross-linked three dimensional biomaterial.

Although progress has been made in recent years to improve the wound healing properties of synthetic matrices, they still do not match the properties of matrices prepared from naturally occurring precursor molecules or polymers.

There exists a need for synthetic matrices that have properties equivalent to those of matrices prepared from naturally occurring materials.

It is therefore an object of the invention to provide matrices, particularly synthetic matrices, that have properties equivalent to matrices prepared from naturally occurring materials, and methods of making and using thereof.

It is therefore an object of the invention to improve the healing capacity of synthetic matrices, in particular for the healing of defects in bone.

It is a further object of the invention to provide synthetic matrices that facilitate the healing of tissue that are not subject to a natural healing response.

It is a further object of the invention to improve the matrix morphology towards optimization of the healing response, in particular to improve the matrix properties with regard to cell infiltration, gelation and degradation time.

### SUMMARY OF THE INVENTION

Methods for making biomaterials for use as wound healing materials and/or tissue regeneration scaffolds, kits containing precursor molecules for forming the biomaterials, and the resulting biomaterials are described herein. The biomaterials are formed from at least a first and a second precursor molecule. The first precursor molecule contains at least two nucleophilic groups, and the second precursor molecule contains at least two electrophilic groups. The nucleophilic and electrophilic groups of the first and second precursor molecules form covalent linkages with each other under physiological conditions. The precursor molecules are selected based on the desired properties of the biomaterial. The sum of the weights of the first and second precursor molecules is in a range from about 8% to about 16% by weight of the composition, preferably from about 10 to about 15%, more preferably from about 12 to about 14.5% by weight of the composition. The ratio of the functional groups of the electrophilic groups (second precursor molecule) and the nucleophilic groups (first precursor molecule) is in the range of between 0.7 and 1.1, preferably between 0.9 and 1.1, more preferably about 1.0 (e.g., stoichiometric ratio). The first and/or second precursor molecule may be covalently bound to one or more molecules selected from the group consisting of cell adhesion peptides, growth factors, and growth factor-like peptides.

The concentration of the precursor molecules in the composition are chosen such that the gelation rate (i.e., time to reach the gel point) and degradation rate of the matrix, as well as its swellability and strength of the matrix are optimized. Preferably the molecular weight of first precursor molecule, the molecular weight of the second precursor molecule and the functionality of the branching points are selected such that the water content of the polymeric network is between 80 and 98% by weight, preferably between 85% and 96% by weight, more preferably of between 87 and 95% by weight of the total weight of the polymeric network after completion of water uptake in the body. In a preferred embodiment, the water content is at its equilibrium weight after completion of water uptake in the body. Completion of water uptake can be achieved either because the equilibrium concentration is reached or because the space available does not allow for further volume increase.

The precursor molecules can be stored separately as dry powders and/or in buffered solutions, typically having an acidic pH. The precursor molecules can be contact for minutes or hours prior to use. In one embodiment, the mixture of the first precursor molecule and second precursor molecule are sprayed together with an activator, a buffer solution having a basic pH, to form the biomaterial with a three dimensional network in situ at the site of need in the body. Alternatively, the two precursor molecules can be mixed using syringe-to-syringe mixing. The combined precursor molecules (plus any additives or biological active agents) are then transferred to a mixing device containing the activator, the basic buffer solution, in a separate compartment, mixed and applied to the site. For compositions with slower gelation times, the precursor molecule and the activator can be mixed ex vivo and applied to the site of administration before substantial crosslinking has occurred.

The biomaterials can be used to induce controlled cell ingrowth and tissues regeneration in a variety of tissues, such as bone. The biomaterials can also be used in wound healing applications.

### BRIEF DESCRIPTION OF THE DRAWINGS

FIG. 1 is a graph showing elastic modulus of hydrogels made by PEG molecules with different structures (i.e. molecular weight and number of arms) and an MMP-sensitive dithiol peptide.

FIG. 2 shows the swelling measurements of hydrogels made by PEG molecules with different structures (i.e. molecular weight and number of arms) and an MMP-sensitive dithiol peptide.

FIG. 3 is a graph showing the swelling (% of initial) versus incubation time for PEG matrices having incorporated therein oligopeptides having different substrate activities.

FIG. 4 is a graph showing the radial invasion (in μm) as a function of incubation time for matrices having different levels of MMP activity,

FIG. 5 is a graph showing the radial invasion rate (μm/hour) as a function of RGD density (μM).

FIG. 6 is a graph showing the radial invasion (μm) versus incubation time for MMP-sensitive hydrogels containing various molecular weights of precursor molecules.

FIG. 7A is a graph showing the of cellular invasion (in mm) within hydrogels that are MMP-sensitive and very loosely cross-linked (i.e. contain a large amount of defects) as a function of incubation time. FIG. 7B is a graph showing radial invasion (as a percentage of the radial invasion of fibrin) for non-degradable materials.

FIG. 8 shows the healing results at 8 weeks in the 8 mm sheep drill defect. Specifically, FIG. 8 shows the percent of the defect filled with calcified tissue for materials with different crosslink densities.

### DETAILED DESCRIPTION OF THE INVENTION

### I. Definitions

“Biomaterial” or “pharmaceutical composition”, as used herein, refers to a material intended to interface with biological systems to evaluate, treat, augment, or replace any tissue, organ or function of the body depending on the material either permanently or temporarily. “Biomaterial”, “matrix”, “hydrogel” and “scaffold” are used synonymously and shall mean a crosslinked polymeric network swollen with water but not dissolved in water, i.e. a hydrogel, which stays in the body for a certain period of time fulfilling certain support functions for traumatized, defective, or injured soft and hard tissue. The term composition refers to the overall composition before it has reached its gel point.

“Biocompatibility” or “biocompatible”, as used herein, refers to the ability of a material to perform with an appropriate host response in a specific application. In the broadest sense, this means a lack of adverse effects to the body in a way that would outweigh the benefit of the material and/or treatment to the patient.

“Strong nucleophile”, as used herein, refers to a molecule which is capable of donating an electron pair to an electrophile in a polar-bond forming reaction. Preferably the strong nucleophile is more nucleophilic than H₂O at physiologic pH. Examples of strong nucleophiles are thiols and amines.

“Electrophilic group” as used herein shall refer to molecule which is capable of accepting an electron pair from a nucleophile in a polar-bond forming reaction. The terms electrophile and electrophilic groups are used synonymously.

“Conjugated unsaturated bond”, as used herein, refers to the alternation of carbon-carbon, carbon-heteroatom or heteroatom-heteroatom multiple bonds with single bonds, or the linking of a functional group to a macromolecule, such as a synthetic polymer or a protein. Such bonds can undergo addition reactions.

“Conjugated unsaturated group”, as used herein, refers to a molecule or a region of a molecule, containing an alternation of carbon-carbon, carbon-heteroatom or heteroatom-heteroatom multiple bonds with single bonds, which has a multiple bond which can undergo addition reactions. Examples of conjugated unsaturated groups include, but are not limited to vinyl sulfones, acrylates, acrylamides, quinones, and vinylpyridiniums, for example, 2- or 4-vinylpyridinium and itaconates.

“Synthetic precursor molecule” or “synthetic precursor molecule”, as used herein, refers to molecules which do not exist in nature.

“Naturally occurring precursor molecule” or “naturally occurring precursor polymer”, as used herein, refers to molecules which can be found in nature.

“Functionalize”, as used herein, means to modify in a manner that results in the attachment of a functional group or moiety. For example, a molecule may be functionalized by the introduction of a molecule which makes the molecule a strong nucleophile or strong electrophile. For example, a molecule, such as PEG, is functionalized to become a thiol, amine, acrylate, or quinone. Proteins may be effectively functionalized by partial or complete reduction of disulfide bonds to create free thiols.

“Functionality”, as used herein, means the number of reactive sites on a precursor molecule.

“Functionality of the branching points”, as used herein, refers to the number of arms extending from one point in the molecule.

“Adhesion site”, as used herein, refers to a peptide sequence to which a molecule, for example, an adhesion-promoting receptor on the surface of a cell, binds. Examples of adhesions sites include, but are not limited to, the RGD sequence from fibronectin, and the YIGSR sequence from laminin. Preferably adhesion sites are incorporated into the biomaterial of the present invention.

“Growth factor binding site”, as used herein, refers to a peptide sequence to which a growth factor, or a molecule(s) which binds a growth factor binds. For example, the growth factor binding site may include a heparin binding site. This site will bind heparin, which will in turn, bind heparin-binding growth factors, for example, bFGF, VEGF, BMP, or TGFβ.

“Protease binding site”, as used herein, refers to a peptide sequence which is a substrate for an enzyme.

“Biological molecule”, as used herein, refers to a molecule that is found in a cell, or in a body, or which can be used as a therapeutic, prophylactic or diagnostic agent for a cell or a body, and which may react with other molecules in its presence. Examples of biological molecules include, but are not limited to, peptides, proteins, nucleic acids, and drugs, such as synthetic, semi-synthetic, or naturally occurring organic and inorganic molecules.

“Regenerate”, as used herein, means to grow back a portion, or all of, a tissue. Tissues which may be regenerated include, but are not limited to, bone, nerve, blood vessel, and cartilage tissue.

“Multifunctional”, as used herein, means more than one electrophilic and/or nucleophilic functional group per molecule (e.g., monomer, oligo- and polymer).

“Self selective reaction”, as used herein, means that the first precursor molecule of the composition reacts much faster with the second precursor molecule of the composition and vice versa than with other compounds present in the mixture and/or at the site of the reaction.

“Respective counterpart” as used herein means the reaction partner of a precursor molecule. The respective counterpart to the electrophilic group is the nucleophilic group and vice versa.

“Self selective reaction” as generally used herein means that the first precursor molecule of the pharmaceutical composition reacts much faster with the second precursor molecule of the pharmaceutical composition and vice versa than with other compounds present both in the pharmaceutical composition and/or at the site of the reaction. As used herein, the nucleophilic group of the first precursor molecule preferentially binds to an electrophilic group of the second precursor molecule rather than to other biological compounds, and an electrophilic group of the second precursor molecule preferentially binds to the nucleophilic group of the first precursor molecule rather than to other biological compounds.

“Cross-linking”, as generally used herein, means the formation of covalent linkages between a precursor molecule containing nucleophilic groups and a precursor molecules containing electrophilic group resulting in an increase in the molecular weight of the material. “Crosslinking” may also refer to the formation of non-covalent linkages, such as ionic bonds, or combinations of covalent and non-covalent bonds.

“Polymeric network”, as used herein, refers to the product of a process in which substantially all of the monomers, oligomers, or polymers are bound by intermolecular covalent linkages through their available functional groups to form a macromolecule.

“Physiological”, as used herein, refers to conditions found in living vertebrates. In particular, physiological conditions refer to the conditions in the human body such as temperature, pH, etc. “Physiological temperatures”, as used herein, refers to a temperature range of between 35° C. to 42° C., preferably around 37° C.

“Crosslink density”, as used herein, refers to the average molecular weight between two crosslinks (M_{c}) of the respective molecules.

“Swelling”, as used herein, refers to the increase in volume and mass due to the uptake of water by the biomaterial. The terms” water-uptake” and “swelling” are used synonymously.

“Gel point” or “gelation” as used herein refers to the point where the viscous modulus and complex modulus cross each other and viscosity increases. Thus the gel point is the stage at which a liquid begins to take on the semisolid characteristics of a gel.

“In situ formation” as generally used herein refers to the ability of mixtures of precursor molecules which are substantially not crosslinked prior to and at the time of injection to form covalent linkages with each other at a physiological temperature at the site of injection in the body.

“Equilibrium state”, as used herein, refers to the state in which a hydrogel undergoes no mass increase or loss when stored under constant conditions in water.

“Weight percent of the precursor molecules”, as used herein, refers to the sum of the weights of the first precursor molecule and the second precursor molecule as a percentage of the weight of the entire composition (% (w/w)). The composition may include solvents, additives, excipients, etc. The weight percent is calculated by adding the weights of the first and second precursor molecules, dividing that sum by the sum of the weights of all of the molecules of the composition, and multiplying by 100. Alternatively, the weight percent can be expressed as a function of the total volume of the composition (% (w/v)). Weight percent by volume is calculated by summing the weights of the first and second precursor molecules, dividing the sum by the total volume of the composition, and multiplying by 100.

### II. Compositions

A pharmaceutical composition for the manufacture of an in situ crosslinkable biomaterials used to induce controlled cell ingrowth and tissue regeneration are described herein. The composition optionally contains additives, colorants, and/or biologically active agents. The biomaterial contains a three-dimensional polymeric network formed from the reaction of at least a first synthetic precursor molecule having n nucleophilic groups and a second precursor molecule having m electrophilic groups wherein the sum of n+m is at least five and wherein the sum of the weights of the first and second precursor molecules is in a range of between about 8% and 16% by weight (weight percentage), preferably from about 10 to about 15% by weight, more preferably from about 12% to about 14.5% by weight of the total composition are described herein.

A. Precursor Molecules

The first precursor molecule contains at least two nucleophilic groups, and the second precursor molecule contains at least two electrophilic groups. The first and second precursor molecules are selected such that the nucleophilic and electrophilic groups form covalent linkages with each other under physiological conditions. This can be achieved via a number of different reaction mechanisms. Examples include nucleophilic substitution reactions, addition reactions, condensation, reactions, and free radical polymerization. In one embodiment, the precursor molecules form a covalent linkage via a Michael addition reaction between nucleophilic moieties on one precursor molecule and conjugated unsaturated moieties on the other molecules. The Michael addition reaction involves the reaction of a nucleophile, such as a thiol, amine, or hydroxyl group, with a conjugated unsaturated moiety, such as an α-unsaturated carbonyl-containing moiety.

The precursor molecules are multifunctional monomers, oligomers and/or polymers. The precursor molecules can be synthetic, semi-synthetic, naturally occurring, or combinations thereof. Preferably the molecular weight of the first precursor molecule is in a range of between 2 to 12 kD, preferably of between 3 to 11 kD and even more preferably of between 5 to 10 kDa. The preferred molecular weight of the second precursor molecule is above the one of the first precursor molecule and preferably in a range of between 10 to 25 kD, even more preferably of between 12 to 20 kD and most preferably of between 14 to 18 kD.

Examples of the first and second precursor molecules include, but are not limited to, proteins, peptides, polyoxyalkylenes, poly(vinyl alcohol), poly(ethylene-co-vinyl alcohol), poly(acrylic acid), poly(ethylene-co-acrylic acid), poly(ethyloxazoline), poly(vinyl pyrrolidone), poly(ethylene-co-vinyl pyrrolidone), poly(maleic acid), poly(ethylene-co-maleic acid), poly(acrylamide), or poly(ethylene oxide)-co-polypropylene oxide) block copolymers. In one embodiment, the first and second precursor molecules are polyethylene glycol modified to contain nucleophilic groups and electrophilic groups, respectively.

The sum of the functionality of the first and second precursor molecule is greater than or equal to 5. In one embodiment, the first precursor molecule has a functionality of four, and the second precursor molecule a functionality of three. In another embodiment, the first precursor molecule has a functionality of 2, and the second precursor molecule a functionality of four. In still another embodiment, both precursor molecules have a functionality of four or more. A small and compact molecule will form a polymeric network with greater strength than an extended molecule, although the functionality, molecular weight and reaction partner might be the same for both molecules. As a general guideline, the ratio of the first and second precursor molecules is selected such that the majority of the functional groups of both molecules react with their respective counterparts. The ratio of the equivalent weights of the electrophilic groups (second precursor molecule) and the nucleophilic groups (first precursor molecule) is in the range of between 0.7 and 1.1, preferably between 0.9 and 1.1, more preferably about 1.0 (i.e., stoichiometric ratio).

a. Nucleophilic Groups

The nucleophilic groups of the first precursor molecule are able to react with electrophilic groups, such as conjugated unsaturated groups in a variety of reaction mechanism, preferably self selectively in the human body through a nucleophilic substitution or Michael type addition reaction. The nucleophiles that are useful are those that are reactive towards conjugated unsaturated groups via addition reactions, in particular in a self-selective Michael-type addition reaction under conditions in the human or animal body. The reactivity of the nucleophile depends on the identity of the unsaturated group. The identity of the unsaturated group is first limited by its reaction with water at physiologic pH. Thus, the useful nucleophiles are generally more nucleophilic than water at physiologic pH. Preferred nucleophiles are commonly found in biological systems, for reasons of toxicology, but are not commonly found free in biological systems outside of cells. Suitable nucleophiles include, but are not limited to, —SH, —NH₂, —OH, —PH₂, and —CO—NH—NH₂. In one embodiment, the nucleophiles are amino or thiol groups.

The usefulness of particular nucleophiles depends upon the situation envisioned and the amount of self-selectivity desired. In the preferred embodiment, the nucleophile is a thiol. However, amines and/or hydroxyl groups may also be effective nucleophiles.

Thiols are present in biological systems outside of cells in paired form, as disulfide linkages. When the highest degree of self-selectivity is desired (e.g. when the gelation reaction is conducted in the presence of tissue and chemical modification of that tissue is not desirable), then a thiol acts as a strong nucleophile.

There are other situations, however, where the highest level of self-selectivity may not be necessary. In these cases, an amine and/or hydroxyl group may serve as an adequate nucleophile. Here, particular attention is paid to the pH, in that the deprotonated amine is a much stronger nucleophile than the protonated amine. Thus, for example, the alpha amine on a typical amino acid (pK as low as 8.8 for asparagine, with an average pK of 9.0 for all 20 common amino acids, except proline) has a much lower pK than the side chain epsilon amine of lysine (pK 10.80). As such, if particular attention is paid to the pK of an amine used as the strong nucleophile, substantial self-selectivity can be obtained. By selection of an amine with a low pK, and then formulation of the final precursor such that the pH is near that pK, one could favor reaction of the unsaturation with the amine provided, rather than with other amines present in the system. In cases where no self-selectivity is desired, the pK of the amine used as the nucleophile is less important. However, to obtain reaction rates that are acceptably fast, the pH is adjusted to be the pH of the final precursor solution so that an adequate number of these amines are deprotonated.

Polyethylene glycol and derivatives thereof can be chemically modified to contain multiple primary amino or thiol groups according to methods set forth, for example, in Chapter 22 of POLY(ETHYLENE GLYCOL) CHEMISTRY: BIOTECHNICAL AND BIOMEDICAL APPLICATIONS, J. Milton Harris, ed., Plenum Press, NY (1992). Polyethylene glycols which have been modified to contain two or more primary amino groups are referred to herein as “multi-amino PEGS.” Polyethylene glycols which have been modified to contain two or more thiol groups are referred to herein as “multi-thiol PEGS.” As used herein, the term “polyethylene glycol(s)” includes modified and or derivatized polyethylene glycol(s), such as block copolymers in which one of the blocks is PEG.

Various forms of multi-amino PEG are commercially available from Nektar Therapeutics, Inc. of San Carlos, Calif. (through its acquisition of Shearwater Polymers of Huntsville, Ala.), and from Texaco Chemical Company of Houston, Tex. under the name “Jeffamine.” Multi-amino PEGs useful in the present invention include Texaco's Jeffamine diamines (“D” series) and triamines (“T” series), which contain two and three primary amino groups per molecule, respectively. Polyamines such as ethylenediamine (H₂N—CH₂CH₂—NH₂), tetramethylenediamine (H₂N—(CH₂)₅—NH₂), pentamethylenediamine (cadaverine) (H₂N—(CH₂)₅—NH₂)—, hexamethylenediamine (H₂N—(CH₂)₆—NH₂), bis(2-hydroxyethyl)amine (HN—(CH₂CH₂OH)₂), bis(2-aminoethyl)amine (HN—(CH₂CH₂NH₂)₂), and tris(2-aminoethyl)amine (N—(CH₂CH₂NH₂)₃) may also be used as the synthetic polymer containing multiple nucleophilic groups.

In one embodiment, the first precursor molecule is a polyethylene glycol having two or more nucleophilic groups. Functionalised polyethylene glycols (PEG) have been shown to combine particularly favourable properties in the formation of synthetic biomaterials. Its high hydrophilicity, its known metabolic pathway and low toxicity make the molecule particularly useful for application in the body. One can readily purchase or synthesize linear (meaning with two ends) or branched (meaning more than two ends) PEGs and then functionalize the PEG end groups according to the reaction mechanisms of choice.

b. Electrophilic Groups

The electrophilic groups on the second precursor molecule are able to form covalent bonds with the nucleophilic groups on the first precursor molecule under physiological conditions. Preferably, the second precursor molecule contains two or more electrophilic groups. In one embodiment, the electrophilic groups are conjugated unsaturated groups.

It is possible to perform nucleophilic addition reactions, in particular Michael addition reactions, on a wide variety of conjugated unsaturated compounds. In the structures shown below, a monomeric, oligomeric or polymeric structure is indicated as P. Various preferred possibilities for the specific identity of P are discussed further herein. P can be coupled to reactive conjugated unsaturated groups, including but not limited to, those structures numbered 1 to 20 in Table 1.

TABLE 1
Molecular structures containing
P and conjugated unsaturated groups

| 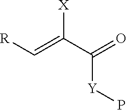 1a | X = H, CH₃, CN, COOW R = H, W, Phenyl- (Ph) Y = NH, O, 1,4-Ph W= C1-C5 aliphatic chain |
|---|---|
| 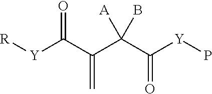 1b | A, B = H, alkyl R = H, alkyl Y = O, NH, 1,4-Ph |
| 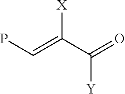 2 | X = CN, COOW Y = OW, Ph W = C1-C5 aliphatic chain |
| 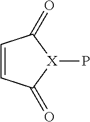 3 | X = N, CH |
| 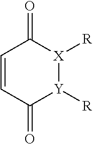 4 | A X = CH Y = CH R = H, W—P (W = NH, O, nihil) B X = N Y = N R = H, P C X—Y = C═C R = W—P (W = NH, O, nihil) |
| 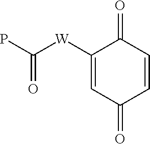 5 | |
| 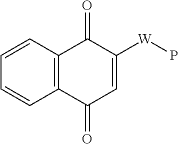 6 | |
| 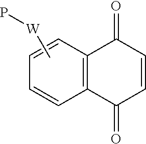 7 | |
| 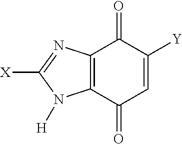 8 | X, Y = H, P P, P P,H P, aliphatic chain |
| 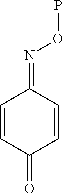 9 | |
| 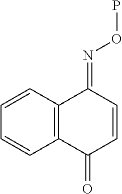 10 | |
| 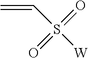 11 | Y = O, NH X = alkali or alkali earth metal ion, P W = P, 1,4-Ph—P |
| 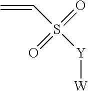 12 | |
| 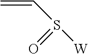 13 | |
| 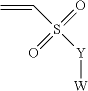 14 | |
| 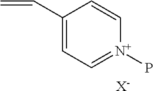 15 | X = halogen, sulphonate |
| 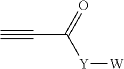 16 | |
| 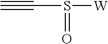 17 | |
| 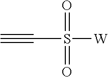 18 | |
| 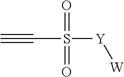 19 | Y = O, NH X = alkali or alkali earth metal ion, P W = P, 1,4-Ph—P |
| 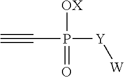 20 | |

Reactive double bonds can be conjugated to one or more carbonyl groups in a linear ketone, ester or amide structure (1a, 1b, 2) or to two in a ring system, as in a maleic or paraquinoid derivatives (3, 4, 5, 6, 7, 8, 9, 10). In the latter case, the ring can be fused to give a naphthoquinone (6, 7, 10) or a 4,7-benzimidazoledione (8) and the carbonyl groups can be converted to an oxime (9, 10). The double bond can be conjugated to a heteroatom-heteroatom double bond, such as a sulfone (11), a sulfoxide (12), a sulfonate or a sulfonamide (13), or a phosphonate or phosphonamide (14). Finally, the double bond can be conjugated to an electron-poor aromatic system, such as a 4-vinylpirydinium ion (15). Triple bonds can be used in conjugation with carbonyl or heteroatom-based multiple bonds (16, 17, 18, 19, 20).

Structures such as 1a, 1b and 2 are based on the conjugation of a carbon-carbon double bond with one or two electron-withdrawing groups. One of them is always a carbonyl, increasing the reactivity passing from an amide, to an ester, and then to a phenone structure. The nucleophilic addition is easier upon decreasing the steric hindrance, or increasing the electron-withdrawing power in the alpha-position. For example, the following relationship exists, CH₃<H<COOW<CN, where CH₃ has the least electron-withdrawing power and CN has the most electron-withdrawing power.

The higher reactivity obtained by using the last two structures can be modulated by varying the bulkiness of the substituents in the beta-position, where the nucleophilic attack takes place; the reactivity decreases in the order P<W<Ph<H. Thus, the position of P can be used to tune the reactivity towards nucleophiles. This family of compounds includes some compounds for which a great deal is known about their toxicology and use in medicine. For example, water-soluble polymers with acrylates and methacrylates on their termini are polymerized (by free radical mechanisms) in vivo. Thus, acrylate and methacrylate-containing polymers have been used in the body in clinical products, but with a dramatically different chemical reaction scheme.

The structures 3-10 exhibit very high reactivity towards nucleophiles, due both to the cis configuration of the double bond and the presence of two electron-withdrawing groups. Unsaturated ketones react faster than amides or imides, due to the stronger electronegativity of these carbonyl groups. Thus, cyclopentendione derivatives react faster than maleimidic ones (3), and para-quinones react faster than maleic hydrazides (4) and cyclohexanones, due to more extended conjugation. The highest reactivity is shown by naphthoquinones (7). P can be placed in positions where it does not reduce the reactivity of the unsaturated group, that is in the opposite part of the ring (3, 5), on another ring (7, 8) or O-linked through a para-quinone mono-oxime (9, 10). To decrease the rate of the nucleophilic addition reaction, P can be also linked to the reactive double bond (6, 8).

The activation of double bonds to nucleophilic addition can be obtained by using heteroatom-based electron-withdrawing groups. In fact, heteroatom-containing analogues of ketones (11, 12), esters and amides (13, 14) provide similar electron-withdrawing behavior. The reactivity towards nucleophilic addition increases with electronegativity of the group. Thus the structures have the following relationship, 11>12>13>14, where 11 is the most electronegative and 14 is the least electronegative. The reactivity towards nucleophilic addition is also enhanced by the linkage with an aromatic ring. A strong activation of double bonds can also be obtained, using electron-withdrawing groups based on aromatic rings. Any aromatic structure containing a pyridinium-like cation (e.g., derivatives of quinoline, imidazole, pyrazine, pyrimidine, pyridazine, and similar sp₂-nitrogen containing compounds) strongly polarizes the double bond and makes possible quick Michael-type additions.

Carbon-carbon triple bonds conjugated with carbon- or heteroatom-based electron-withdrawing groups can easily react with sulfur nucleophiles, to give products from simple and double addition. The reactivity is influenced by the substituents, in a manner similar to double bond-containing analogous compounds discussed above. In a preferred embodiment, the electrophilic groups are acrylate groups.

Polyethylene glycol can be chemically modified to contain multiple electrophilic groups according to methods set forth, for example, in Chapter 22 of POLY(ETHYLENE GLYCOL) CHEMISTRY: BIOTECHNICAL AND BIOMEDICAL APPLICATIONS, J. Milton Harris, ed., Plenum Press, NY (1992). Various forms of multi-electrophilic PEG are commercially available from Nektar Therepeutics, Inc. of San Carlos, Calif. (through its acquisition of Shearwater Polymers of Huntsville, Ala.).

The formation of ordered aggregates (liposomes, micelles) or the simple phase separation in an aqueous environment increases the local concentration of unsaturated groups and thus, the reaction rate. In this case, the latter depends also on the partition coefficient of the nucleophiles, which increases for molecules with enhanced lipophilic character.

In a preferred embodiment, the matrix is formed by the reaction of a four-arm polyoxyalkylene molecule as the first precursor molecule end-functionalized at each of the arms with an electophilic group and the second precursor molecule is a linear bifunctional polyoxyalkylene end-functionalized with nucleophilic groups wherein the sum of the weight of the first and second precursor molecule is in a range of between 8 to 16 weight %, preferably 10 to 15 weight %, more preferably between 12 and 14.5 weight % of the total weight of the composition (before network formation). The first precursor molecule typically has a molecular weight of between 1 to 4.5 kDa, preferably 1.5 to 4 kDa, more preferably about 3.4 kDa and the second precursor molecule has a molecular weight of between 10 to 20 kDa, more preferably about 15 kD.

If the first precursor molecule is a three or four arm polymer with a functional group at the end of each arm and the second precursor molecule is a linear bifunctional molecule, then the molecular weight of the arms of the first precursor molecule and the molecular weight of the second precursor molecule are preferably chosen such that the links between the branching points after formation of the network have a molecular weight in the range of between 10 to 13 kD (under the conditions that the links are linear, not branched), preferably between 11 and 12 kD. This allows for a starting concentration of the sum of first and second precursor molecules in a range of between 8 to 16 weight %, preferably 10 to 15 weight % and even more preferably between 12 and 14.5 weight % of the total weight of the composition (before network formation). If the branching degree of the first precursor molecule is increased to eight and the second precursor molecule is still a linear bifunctional molecule, the molecular weight of the links between the branching points is preferably increased to a molecular weight of between 18 to 24 kD. If the branching degree of the second precursor molecule is increased from linear to a three or four arm precursor molecule the molecular weight, i.e. the length of the links increase accordingly.

In another embodiment, the first precursor molecule is a trifunctional three arm 15 kD polymer, i.e. each arm having a molecular weight of 5 kD and the second precursor molecule is a bifunctional linear molecule having molecular weight in the range of between 0.5 to 1.5 kD, more preferably around lkD. The first and second precursor molecules are preferably polyethylene glycol.

B. Active Agents

The biomaterial may also contain bioactive agents, such small molecules or peptides proteins which can diffuse slowly from the biomaterial and thus helping the tissue to regenerate and heal. In such cases, the biomaterial works as both a tissue regeneration scaffold/wound healing material and as a drug delivery matrix. The bioactive factors and/or small molecules can simply be mixed into the biomaterial or can be covalently bound to the biomaterial and released by hydrolytic or enzymatic degradation.

Cell adhesion peptides, growth factors, and growth factor-like peptides can be incorporated into the composition. The peptides can be mixed with the precursor molecules or can be covalently coupled to the precursor molecules.

For example, peptides that induce cell adhesion through specific receptor-ligand binding and molecules that enable the matrix to undergo cell-triggered remodeling by matrix metalloproteinases (MMP) can be incorporated into the compositions. MMPs are major proteins in mammalian tissues and degradation of MMP substrates plays an important role in natural ECM turnover (e.g. during wound healing) and tissue regeneration. Other enzyme classes may also be targeted by incorporation of a substrate in the composition that is specific for the particular enzymes that is desired. In these hydrogels, the mechanism and speed at which cells migrate in three dimensions both in vitro and in vivo can be readily controlled by the characteristics and composition of the matrix independent of addition of any free or matrix-associated exogenous signaling molecules such as growth factors or cytokines.

In one embodiment, a peptide that is a protease substrate is one of the precursor molecules so as to make the network capable of being infiltrated and degraded by cells. One of ordinary skill in the art can readily synthesize peptides that contain two or more cysteine residues, and this molecule can as a precursor molecule containing nucleophilic groups. For example, a peptide with two free cysteine residues will readily form a hydrogel when mixed with a three or four arm end-functionalized 15 to 20 k polyethylene glycol (PEG) acrylate at physiological or slightly higher pH (e.g., 8 to 9; the gelation will also proceed higher pHs, but at the potential expense of self-selectivity). When the first and second precursor molecules are mixed together in liquid form, they react over a period of a few minutes to form an elastic gel, consisting of a network of PEG chains, bearing the nodes of the network, with the peptides as connecting links The gelation is self-selective, meaning the peptide reacts mostly with the PEG molecule and no other molecules, and the PEG molecule reacts mostly with the peptide and no other molecules. In still another embodiment bifunctional agents can be incorporated to provide chemical bonding to other species (e.g., a tissue surface).

In another embodiment, peptide sites for cell adhesion are incorporated into the matrix, such as peptides that bind to adhesion-promoting receptors on the surfaces of cells. Examples of adhesion promoting peptides include, but are not limited to, RGD sequence from fibronectin and the YIGSR sequence from laminin. For example, the adhesion promoting peptides can be covalently coupled to one of the precursor molecules. This can be done, for example, simply by mixing a cysteine-containing peptide with the precursor molecule containing electrophilic groups, such as PEG diacrylate or triacrylate, PEG diacrylamide or triacrylamide or PEG diquinone or triquinone a few minutes before mixing with the remainder of the precursor molecule containing the nucleophilic groups.

In yet another embodiment, growth factors or growth factor like peptides are covalently attached to one of the precursor molecules or physically incorporated into the biomaterial. Examples of classes of growth factors and growth-factor like peptides include, but are not limited to, TGF β, BMPs, IGFs, and PDGFs. Examples of specific growth factors or growth factor-like peptides include, but are not limited to, BMP 2, BMP 7, TGF β1, TGF β3, IGF 1, PDGF AB, human growth releasing factor, PTH 1-84, PTH 1-34 and PTH 1-25. PTH (PTH 1-84, PTH 1-34 and PTH 1-25) showed particularly good bone formation when covalently bound to a synthetic matrix. Best results are achieved by covalently binding PTH 1-34 (amino acid sequence SVSEIQLMHNLGKHLNSMERV EWLRKKLQDVHNF) to a synthetic matrix capable of being infiltrated by cells and afterwards degraded. The growth factors or growth factor like peptides are recombinantly expressed or chemically synthesized with at least one additional cysteine group, containing a free thiol group (i.e., —SH) either directly attached to the protein or peptide or through a linker sequence. The linker sequence can additionally comprise an enzymatically degradable amino acid sequence, such as a plasmin degradable sequence, so that the growth factor can be cleaved of from the matrix by enzymes in substantially the native form. For example, the sequence GYKNR is a plasmin degradable sequence. The growth factor or growth factor-like peptide can be coupled to the matrix by attaching an additional amino acid sequence to the N-terminus of the molecules that contains at least one cysteine. The thiol group of the cysteine can react with an electrophilic group, such as a conjugated unsaturated group, on the other precursor molecule to form a covalent linkage.

One of ordinary skill in the art can readily determine the concentration of adhesion peptides and/or growth factors or growth factor-like peptides, the density of these materials on the matrix, and the kinetic degradability of peptides containing protease sequences which is best suited for a particular application.

C. Additives

The pharmaceutical composition may further contain organic and/or inorganic additives, such as thixotropic agents, radiopaque or fluorescent agents in order to track the performance of application or to instantaneously detect potential leakage if not readily visible, stabilizers for stabilization of the precursor molecules like radical scavengers to avoid premature polymerization like butylated hydroxytoluene or dithiothreitol and/or fillers which can result in an increase of the mechanical properties (ultimate compressive strength and Young's modulus E) of the biomaterial compared to the mechanical properties of the polymeric network. Depending on the application the pharmaceutical composition (and thus biomaterial) it may contain a colorant, preferably an organic one. In one embodiment methylene blue is added as a colorant. Methylene blue not only acts as a colorant but also as a stabilizer to thiol containing precursor molecules acting as a reducing agent and as an indictor for disulfide formation (since it becomes colorless upon reduction). Another preferred colorant is lissamine green.

D. Bases

The in situ crosslinking of the first and the second precursor molecules takes place under basic but still physiological conditions. A variety of bases, comply with the requirements of catalyzing the reaction under physiological conditions and of not being detrimental to the patient's body and thus acting as activators in the formation of the biomaterial Suitable bases include, but are not limited to, tertiary alkyl-amine, such as tributylamine, triethylamine, ethyldiisopropylamine, or N,N-dimethylbutylamine. For a given pharmaceutical composition (and mainly dependent on the type of precursor molecules), the gelation time is dependant on the amount and type of base. Thus the gelation time of the pharmaceutical composition can be controlled and adjusted to the need of application by varying the base concentration and the type of base. In a preferred embodiment the base, as the activator of the covalent crosslinking reaction, is selected from aqueous buffer solutions which have their pH and pK value in the same range. The pK range being preferably in between 9 and 13. If the base has two pK values in the basic range the first one is preferably between 8.5 and 10 whereas the second one is between 10 and 13. Sodium carbonate, sodium borate and glycine are preferred examples.

### III. Biomaterials

The properties of the matrices are dependent on concentration of the precursor molecules in the composition. By choosing a weight range of the combined precursor molecules from 8 to 16 weight %, preferably 10 to 15 weight %, more preferably from 12 to 14.5 weight % of the total weight of the composition, the gelation rate and degradation rate of the matrix, as well as its swellability and strength, can be optimized. If the concentration of the precursor molecules is too low, the rate at which the precursor molecules crosslink to form a hydrogel under physiological conditions is too slow for medical applications and the degradation rate of the biomaterial in vivo is too fast to achieve a meaningful healing response. If the concentration of the precursor molecules extend above the ideal range, the swelling of the matrices in the body can become excessive, thus building up pressure on the surrounding tissue. The inability of the material to swell due to pressure from the surrounding tissue can inhibit the healing response since cells and other materials needed for healing cannot penetrate the matrix.

For most healing indications, the rate of cell ingrowth or migration of cells into the matrix in combination with the degradation rate of the matrix is crucial for the overall healing response. The potential of matrices to become invaded by cells is primarily a question of network density, i.e. the space between branching points or nodes. If the existing space is too small in relation to the size of the cells or if the rate of degradation of the matrix, which results in creating more space within the matrix, is too slow, a very limited healing response will be observed. Healing matrices found in nature, as e.g. fibrin matrices, which are formed as a response to injury in the body are known to consist of a network which is an optimal substrate for cell invasion. The infiltration can be promoted by ligands for cell adhesion which are an integrated part of the fibrin network.

If n and/or m of the first and/or second precursor molecule are greater than two, than the molecular weight of the arms for a given precursor are substantially similar to each other. “Substantially similar”, as used herein, means that the molecular weights of the arms for a given precursor are with ±10 weight % of each other. In one embodiment, the molecular weigh of the arms of the precursor molecules are identical. The ratio of the nucleophilic and electrophilic groups of the of the first and second precursor molecules is preferably between 0.9 and 1.1, preferably the ratio is 1 and thus no functional groups are left unreacted.

Preferably the molecular weight of the arms of the first precursor molecule, the molecular weight of the second precursor molecule and the functionality of the branching points are selected such that the water content of the polymeric network is between 80 and 98% by weight, preferably between 85% and 96% by weight, more preferably of between 87 and 95% by weight of the total weight of the polymeric network after completion of water uptake in the body. In a preferred embodiment, the water content is at its equilibrium weight after completion of water uptake uptake in the body. Completion of water uptake can be achieved either because the equilibrium concentration is reached or because the space available does not allow for further volume increase.

Matrices made from synthetic hydrophilic precursor molecules, like functionalized polyethylene glycol, swell in an aqueous environment after formation of the polymeric network. In order to achieve a sufficiently short gelling time (between 3 to 15 minutes, preferably 3 to 10 minutes, more preferably 5-10 minutes) under physiological conditions (e.g., pH up to 8, preferably between 7 and 8 and a temperature between 36 and 38° C.) the starting concentration of the precursor molecules have to be in an optimal concentration range. Swelling of the polymeric network is important to enlarge and widen the space between the branching points in order to facilitate cell migration.

Irrespective of the starting concentration of the precursor molecules, hydrogels made from the same synthetic precursor molecules swell to the same water content in equilibrium state. This means that the higher the starting concentration of the precursor molecules are, the higher the end volume of the matrices is when it reaches its equilibrium state. If the space available in the body is too small to allow for sufficient swelling of the matrix, the rate of cell infiltration and as a consequence the healing response will decrease. As a consequence, the optimum between three contradictory requirements for application in the body must be found. On the one hand, the starting concentrations must be sufficiently high to guarantee the necessary gelling-time. This may result, however, in matrices which require more space than is available in the defect to achieve the necessary water content and thus remain too dense for cell infiltration and have degradation rates that are too long.

The relation between matrix degradation and cell infiltration can be manipulated by (1) varying the structure (i.e. the chain length and number of arms) of the precursor polymer for cell infiltration; (2) varying the affinity and concentration of adhesion ligands covalently bound to the network to increase cell infiltration; (3) varying, in the case of enzymatically degradable gels, the specificity of the protease substrate to degradation by a desired protease secreted by cells and the enzymatic activity (Km/kcat) or kinetics of enzymatic hydrolysis of the employed protease substrate; (4) varying, in the case of hydrolytically degradable gels, the susceptibility of the matrix to hydrolysis under physiological conditions; and (5) covalently coupling to the matrix molecules that upregulate or downregulate the expression and secretion of matrix metalloproteases (MMPs) (e.g. growth factors). These factors are largely independent of the crosslinking chemistry used to form the biomaterial (i.e., whether the precursor molecules are crosslinked using Michael Addition, substitution, addition, or condensation chemistry).

The reaction mechanism for producing the three dimensional network can be chosen among various reaction mechanisms known in the art, such as substitution reactions, condensation reactions, free radical reaction and addition reactions. In the case of substitution, condensation and addition reactions, one of the precursor molecules contains nucleophilic groups and the other precursor molecule contains electrophilic groups, preferably conjugated unsaturated groups or bonds. In the case of free radical reactions, both precursor molecules comprise unsaturated bonds, preferably conjugated unsaturated bonds.

A particularly preferred reaction mechanism is the Michael type addition reaction between a conjugated unsaturated group or bond and a strong nucleophile as described in WO 00/44808. For Michael type addition reactions, the first precursor molecule preferably contains amino or thiol-groups and the second precursor molecule preferably contains conjugated unsaturated groups, such as vinylsulfone- or acrylate groups. End-linking of the two precursor molecules yields a stable three-dimensional network. This Michael-type addition to conjugated unsaturated groups takes place in quantitative yields under physiological conditions without creating any byproducts.

### IV. Methods of Making

A. Storage

The first and second precursor molecules are preferably stored under exclusion of oxygen and light and at low temperatures, e.g., around +4° C., to avoid decomposition of the functional groups prior to use. The precursors can be stored as dry powders or in buffered solutions. If the precursor molecules are stored in the buffer solution, the pH of the solution is typically acidic, e.g., around 5.5. The content of functional groups of each precursor molecule is measured immediately prior to use and the ratio of first and second precursor molecule (and other precursor molecule when appropriate) is adjusted according to the predetermined equivalent weight ratio of the functional groups.

B. Preparation of Compositions for Wound Healing and Tissue Regeneration

In order to form the biomaterial, the first and the second precursor molecules can be dissolved in a solution containing a base. Alternatively, precursor molecule solutions can be mixed with a buffer solution having a basic pH. For example, the precursor molecules and base/buffer solution can be stored separately in bipartite syringes which have two chambers separated by an adjustable partition rectangular to the syringe body wall. One of the chambers can contain the precursor molecule in solid pulverized form, the other chamber contains an appropriate amount of base/buffer solution. If pressure is applied to one end of the syringe body, the partition moves and releases bulges in the syringe wall releasing buffer into the chamber containing the corresponding precursor molecule which upon contact with the base/buffer solution dissolves to form a solution. A bipartite syringe body is used for storage and dissolution of the other precursor molecule in the same way. If both precursor molecules are dissolved, both bipartite syringe bodies are attached to a two way connecting device and the contents are mixed by squeezing them through the injection needle attached to the connecting device. The connecting device additionally can comprise a static mixer to improve mixing of the contents. The mixed molecules are injected directly at the site of need in the body by connecting the static mixer to the injection needle or the mixture is squeezed in a further syringe which then is connected to the injection needle.

In one embodiment, a solution of bioactive peptides, for example binders to adhesion-promoting receptors on the cell surface flanked by a single cysteine and/or growth factors or growth factor like peptides, is reacted with the precursor molecule comprising conjugated electrophilic groups in order to covalently couple the peptides to the precursor molecule. In the second step, a hydrogel is formed by mixing the peptide-grafted precursor solution with a solution containing the precursor molecule containing nucleophilic groups. The crosslinking reaction is self-selective; very few extracellular proteins contain free thiols and 1,4-conjugated unsaturations are rarely found in biological environments allowing gels to be formed in situ and directly at a surgical site in the presence of other proteins, cells and tissues.

The starting concentration of the first and second precursor molecule is in a range of 8 to 16 weight %, preferably 10 to 15 weight %, more preferably between 12 and 14.5 weight % of the total weight of the composition (before network formation). All molecules are sterilized prior to mixing. This preferably is done by sterilfiltration of the precursor molecules and gamma irradiation of the additives/fillers.

### V. Kits for Forming In Situ Crosslinkable Compositions

The kit contains at least a first and a second precursor molecule. The kit may also contain one or more devices, such as syringes, for administering the first and second molecules. The kit may contain a base and/or buffers for polymerizing the precursor molecule. Optionally, the first and/or the second precursor molecule(s) contain one or more additives and/or biologically active agents, such as cell adhesion peptides, growth factors, and growth factor-like peptides. The active agents may be mixed with the first and/or second precursor molecules or can be covalently bound to the first or second precursor molecule. In one embodiment, one or both of the precursor components is covalently bound to one or more cell adhesion peptides, growth factors, growth factor-like peptides, and combinations thereof. The precursor molecules may be placed in the one or more devices prior to administration to a patient.

### VI. Method of Use

A. Wound Healing/Tissue Regeneration

The multifunctional precursor molecules are selected and tailored to produce biomaterials with the desired properties. The precursor molecules are capable of in situ crosslinking under physiological conditions to specific would healant and/or tissue injury/defect requirements. In the preferred embodiment, the biomaterials are used to induce controlled cell ingrowth and tissue regeneration in a variety of tissues, such as bone. The compositions may contain one or more active agents which are released from the matrix to aid in wound healing.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### EXAMPLES

### Example 1

### Preparation of Basic Reagents

Preparation of PEG-Vinylsulfones

Commercially available branched polyethylene glycols (PEGs) (4arm PEG, mol. wt. 14,800, 4arm PEG, mol. wt. 10,000 and 8arm PEG, mol. wt. 20,000; Shearwater Polymers, Huntsville, Ala., USA) were functionalized at the OH-termini.

PEG vinyl sulfones were produced under argon atmosphere by reacting a dichloromethane solution of the precursor polymers (previously dried over molecular sieves) with NaH and then, after hydrogen evolution, with divinylsulfone (molar ratios: OH 1:NaH 5:divinylsulfone 50). The reaction was carried out at room temperature for 3 days under argon with constant stirring. After the neutralization of the reaction solution with concentrated acetic acid, the solution was filtered through paper until clear. The derivatized polymer was isolated by precipitation in ice cold diethylether. The product was redissolved in dichloromethane and reprecipitated in diethylether (with thoroughly washing) two times to remove all excess divinylsulfone. Finally the product was dried under vacuum. The derivatization was confirmed with ¹H NMR. The product showed characteristic vinyl sulfone peaks at 6.21 ppm (two hydrogens) and 6.97 ppm (one hydrogen). The degree of end group conversion was found to be 100%.

Preparation of PEG-Acrylates

PEG acrylates were produced under argon atmosphere by reacting an azeotropically dried toluene solution of the precursor polymers with acryloyl chloride, in presence of triethylamine (molar ratios: OH 1:acryloyl chloride 2:triethylamine 2.2). The reaction proceeded with stirring overnight in the dark at room temperature. The resulting pale yellow solution was filtered through a neutral alumina bed; after evaporation of the solvent, the reaction product was dissolved in dichloromethane, washed with water, dried over sodium sulphate and precipitated in cold diethyl ether. Yield: 88%; conversion of OH to acrylate: 100% (from ¹H-NMR analysis)

¹H-NMR (CDCl₃): 3.6 (341H (14800 4arm: 337H theor.), 230 (10000 4arm: 227H theor.), or 210H (20000 8arm: 227H theor.), PEG chain protons), 4.3 (t, 2H, —CH₂—CH₂—O—CO—CH═CH₂), 5.8 (dd, 1H, CH₂═CH—COO—), 6.1 and 6.4 (dd, 1H, CH₂═CH—COO—) ppm.

FT-IR (film on ATR plate): 2990-2790 (υ C—H), 1724 (υ C═O), 1460 (υₛ CH₂), 1344, 1281, 1242, 1097 (υₐₛ C—O—C), 952, 842 (υₛ C—O—C) cm⁻¹.

Peptide Synthesis

All peptides were synthesized on solid resin using an automated peptide synthesizer (9050 Pep Plus Synthesizer, Millipore, Framingham, USA) with standard 9-fluorenylmethyloxycarbonyl chemistry. Hydrophobic scavengers and cleaved protecting groups were removed by precipitation of the peptide in cold diethyl ether and dissolution in deionized water. After lyophilization, the peptides were redissolved in 0.03 M Tris-buffered saline (TBS, pH 7.0) and purified using HPLC (Waters; Milford, USA) on a size exclusion column with TBS, pH 7.0 as the running buffer.

### Example 2

### Hydrogel Formation by Conjugate Addition Reactions

MMP-Sensitive Gels Formed by Conjugate Addition with a Peptide-Linked Nucleophile and a PEG-Linked Conjugated Unsaturation that Allow Proteolytic Cell Migration

The synthesis of gels is accomplished entirely through Michael-type addition reaction of thiol-PEG onto vinylsulfone-functionalized PEG. In a first step, adhesion peptides were attached pendantly (e.g. the peptide Ac-GCGYGRGDSPG-NH₂) to a multiarmed PEG-vinylsulfone and then this precursor was cross-linked with a dithiol-containing peptide (e.g. the MMP substrate Ac-GCRDGPQGIAGFDRCG-NH₂). In a typical gel preparation for 3-dimensional in vitro studies, 4arm-PEG-vinylsulfone (mol. wt. 15000) was dissolved in a TEOA buffer (0.3M, pH 8.0) to give a 10% (w/w) solution. In order to render gels cell-adhesive, the dissolved peptide Ac-GCGYGRGDSPG-NH₂ (same buffer) were added to this solution. The adhesion peptide was allowed to react for 30 minutes at 37° C. Afterwards, the crosslinker peptide Ac-GCRDGPQGIWGQDRCG-NH₂ was mixed with the above solution and gels were synthesized. The gelation occurred within a few minutes, however, the crosslinking reaction was carried out for one hour at 37° C. to guarantee complete reaction.

MMP-Non-Sensitive Gels Formed by Conjugate Addition with a PEG-Linked Nucleophile and a PEG-Linked Conjugated Unsaturation that Allow Non-Proteolytic Cell Migration

The synthesis of gels is also accomplished entirely through Michael-type addition reaction of thiol-PEG onto vinylsulfone-functionalized PEG. In a first step, adhesion peptides were attached pendantly (e.g. the peptide Ac-GCGYGRGDSPG-NH₂) to a multiarmed PEG-vinylsulfone and then this precursor was crosslinked with a PEG-dithiol (m.w. 3.4 kD). In a typical gel preparation for 3-dimensional in vitro studies, 4arm-PEG-vinylsulfone (mol. wt. 15000) was dissolved in a TEOA buffer (0.3M, pH 8.0) to give a 10% (w/w) solution. In order to render gels cell-adhesive, the dissolved peptide Ac-GCGYGRGDSPG-NH₂ (in same buffer) were added to this solution. The adhesion peptide was allowed to react for 30 minutes at 37° C. Afterwards, the PEG-dithiol precursor was mixed with the above solution and gels were synthesized. The gelation occurred within a few minutes, however, the crosslinking reaction was carried out for one hour at 37° C. to guarantee complete reaction.

### Example 3

### Hydrogel Formation by Condensation Reactions

MMP-Sensitive Gels Formed by Condensation Reactions with a Peptide X-Linker Containing Multiple Amines and a Electrophilically Active PEG that Allow Proteolytic Cell Migration

MMP-sensitive hydrogels were also created by conducting a condensation reaction between MMP-sensitive oligopeptide containing two MMP substrates and three Lys (Ac-GKGPQGIAGQKGPQGIAGQKG-NH₂) and a commercially available (Shearwater polymers) difunctional double-ester PEG-N-hydroxysuccinimide (NHS-HBS-CM-PEG-CM-HBA-NHS). In a first step, an adhesion peptides (e.g. the peptide Ac-GCGYGRGDSPG-NH₂) was reacted with a small fraction of NHS-HBS-CM-PEG-CM-HBA-NHS and then this precursor was cross-linked to a network by mixing with the peptide Ac-GKGPQGIAGQKGPQGIAGQKG-NH₂ bearing three ε-amines (and one primary amine). In a typical gel preparation for 3-dimensional in vitro studies, both molecules were dissolved in 10 mM PBS at pH7.4 to give a 10% (w/w) solution and hydrogels were formed within less then one hour.

In contrast to the present hydrogels formed by Michael-type reaction, the desired self-selectivity in this approach is not guaranteed, since amines present in biological materials like cells or tissues will also react with the difunctional activated double esters. This is also true for other PEGs bearing electrophilic functionalities such as PEG-oxycarbonylimidazole (CDI-PEG), or PEG nitrophenyl carbonate.

MMP-Non-Sensitive Hydrogels Formed by Condensation Reactions with a PEG-Amine Cross-Linker and a Electrophilically Active PEG that Allow Non-Proteolytic Cell Migration

Hydrogels were also formed by conducting a condensation reaction between commercially available branched PEG-amines (Jeffamines) and the same difunctional double-ester PEG-N-hydroxysuccinimide (NHS-HBS-CM-PEG-CM-HBA-NHS). In a first step, the adhesion peptides (e.g. the peptide Ac-GCGYGRGDSPG-NH₂) was reacted with a small fraction of NHS-HBS-CM-PEG-CM-HBA-NHS and then this precursor was cross-linked to a network by mixing with the multiarm PEG-amine. In a typical gel preparation for 3-dimensional in vitro studies, both molecules were dissolved in 10 mM PBS at pH7.4 to give a 10% (w/w) solution and hydrogels were formed within less then one hour.

Again, in contrast to the present hydrogels formed by Michael-type reaction, the desired self-selectivity in this approach is not guaranteed, since amines present in biological materials like cells or tissues will also react with the difunctional activated double esters. This is also true for other PEGs bearing electrophilic functionalities such as PEG-oxycarbonylimidazole (CDI-PEG), or PEG nitrophenyl carbonate.

### Example 4

### Equilibrium Swelling Measurements of Hydrogels Made by Conjugate Addition with Various Macromers and a Thiol-Containing MMP-Sensitive Peptide

Hydrogel structure-function studies were conducted in order to test whether a connection between precursor parameters and network properties could be established and attributed to the well-characterized microstructure of the gels.

Hydrogel Formation and Equilibrium Swelling Measurements

Gels were weighted in air and ethanol before and after swelling and after freeze-drying using a scale with a supplementary density determination kit. Based on Archimedes' buoyancy principle the gel volume after cross-linking and the gel volume after swelling was calculated. Samples were swollen for 24 hours in distilled water. The crosslink density and the molecular weight between cross-links (M_{c}) were calculated based on the model of Flory-Rehner and its modified version by Peppas-Merrill.

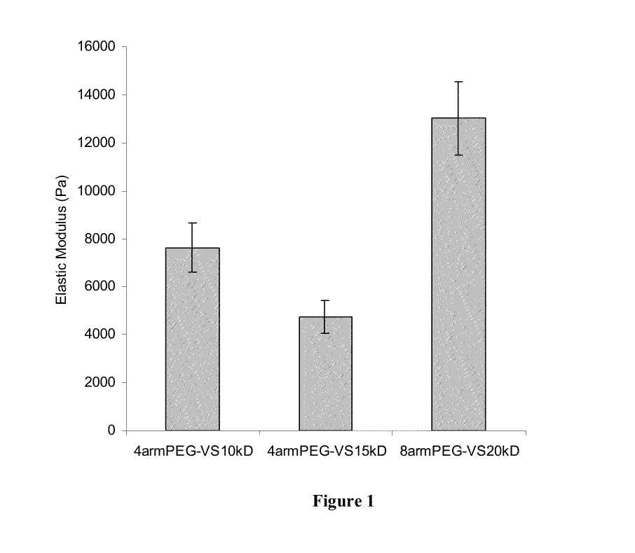

PEG macromer structure (i.e. molecular weight. and number of arms) directly correlates with swelling characteristics of the networks. The swelling ratio increased with a decrease of the arm length or an increase in functionality of the X-linker By changing the chain length and number of arms of the macromers at constant precursor concentration (10% w/w), the swelling ratio (and thus the X-link density and molecular weight between X-links) were significantly altered (FIG. 1). For example, the elastic modulus G′ increased with a decrease of the arm length or an increase in functionality of the crosslinking sites. The correlation between precursor parameters and network properties can be attributed to the well-characterized microstructure of the hydrogels.

### Example 5

### Viscoelastic Measurements of Hydrogels Made by Conjugate Addition with Various Macromers and a Thiol-Containing MMP-Sensitive Peptide

Dynamic viscoelastic properties of hydrogels were studied via small strain oscillatory shear experiments using a Bohlin CVO 120 High Resolution rheometer with plate-plate geometry at 37° C. and pH 7.4 under humidified atmosphere between the plates. The PEG-multiacrylate and peptide precursor solutions (30 μl each) were applied to the bottom plate and briefly mixed with a pipette tip. The upper plate (20 mm diameter) was then immediately lowered to a measuring gap size of 0.1 mm. After a short pre-shear period (to ensure mixing of the precursors), the dynamic oscillating measurement was started. The evolution of the storage (G′) and loss (G″) moduli and phase angle (δ) at a constant frequency of 0.5 Hz was recorded. An amplitude sweep was performed in order to confirm that the parameters (frequency and strain) were within the linear viscoelastic regime. PEG macromer structure (i.e. molecular weight and number of arms) directly correlates with viscoelastic characteristics of the networks

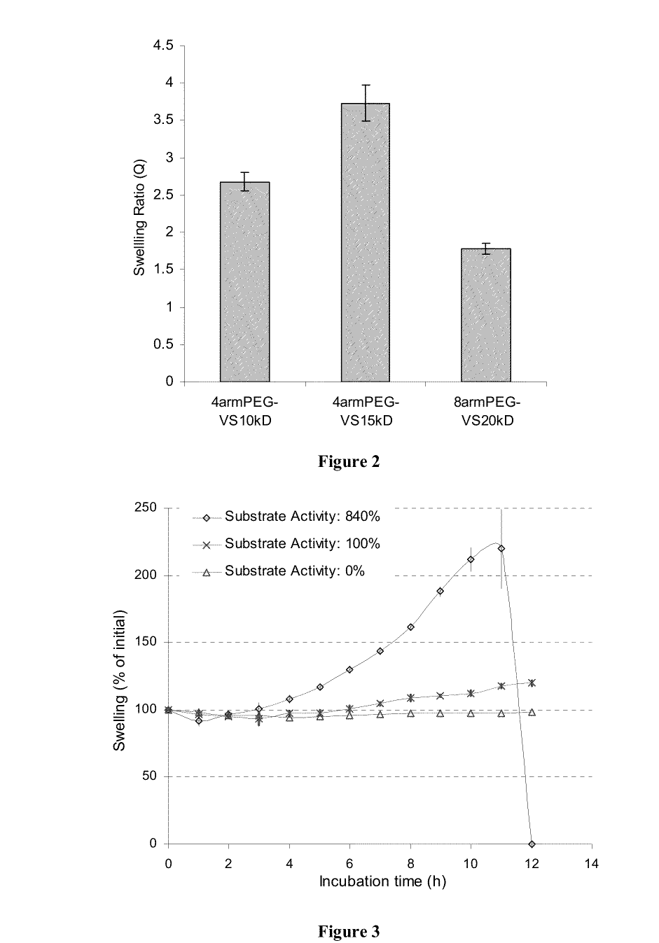

By changing the chain length and number of arms of the macromers at constant precursor concentration (e.g. 10% w/w), the shear moduli (G′ and G″) were significantly altered and G′ increased with a decrease of the arm length or an increase in functionality of the X-linker again implying that there is a clear correlation between precursor parameters and network properties that can be attributed to the well-characterized microstructure of the gels (FIG. 2). FIG. 2 shows that the swelling ratio increases as the molecular weight of the arms decrease or the degree of functionality increases.

### Example 6

### Biochemical Degradation by Human MMP-1 of Gels Formed by Conjugate Addition with Peptides Containing Two Cysteine Residues with MMP Substrate Sequences of Various Enzymatic Activity

Enzymatic degradation was assessed biochemically by exposure of MMP-sensitive hydrogels to the proteolytic action of activated MMP-1. Hydrogels bearing substrates with three different enzymatic activity were tested (K_{M}/k_{cat}=840%, 100%, 0%). Degradation of hydrogels by MMP-1 was determined by measuring the change of swelling during degradation.

Demonstration of MMP-Degradability and its Sensitivity to the Enzymatic Activity of the Incorporated Oligopeptides

Degradation kinetic (swelling, i.e. weight change) of hydrogels containing MMP-substrates with different activity responded to the amino acid sequence of the protease substrate peptide (i.e. the enzymatic activity). Thus, the kinetics of proteolytic gel breakdown can be engineered by very simple means (FIG. 3). FIG. 3 shows MMP-degradability and its dependence on enzymatic activity of the incorporated oligopeptides. The degree of swelling is measured as a function of incubation time.

### Example 7

### Embedding and Culture of hFF-Fibrin Clusters Inside Synthetic PEG-Based Hydrogels to Assess Three-Dimensional Cell Invasion Capacity of the Matrix

Near-confluent cultures of human foreskin fibroblasts (hFFs) were trypsinized, centrifuged and resuspended in 2% (m/v) fibrinogen from human plasma (Fluka, Switzerland) in sterile phosphate buffered solution (PBS) to a concentration of 30000 cells/4. To induce gelation of the hFF-fibrinogen suspension, thrombin (Sigma T-6884, Switzerland) and Ca⁺⁺ were added to final concentrations of 2 NIH units/mL and 2.5 mM, respectively, and rapidly mixed with the cell suspension. Prior to gelation, 2 μL droplets of this cell-fibrinogen precursor were gelled on microscope slides for ca. 15 min. at 37° C. The hFF-fibrin clusters were embedded inside 25 μl PEG-based hydrogels by placing three to four clusters into precursor solution prior to gelation. Such hFF-fibrin clusters embedded inside the PEG-based hydrogels were cultured serum-containing DMEM in the 12-well tissue culture plates for up to 30 days. Cell invasion from the cluster into the synthetic gel matrix was imaged and recorded with their center plane in focus. To quantify the penetration depth of the outgrowth, the area of the original hFF-fibrin cluster was measured in the center plane, as was the area of the hFF outgrowth, defined by the tips of the hFF branches in the center plane of focus. These two areas were approximated as circular areas, and their theoretical radii subtracted from each other to give an average hFF outgrowth length.

The fact that cells grow out from the clusters implies that Michael-type addition to conjugated unsaturated groups is self-selective, i.e. acrylates or vinylsulfone react with thiols much faster than with amines that are present on the cell surface. Thus, such materials can be used clinically for example to fill tissue defects by in situ gelation.

### Example 8

### Changing Cell Invasion Rate into MMP-Sensitive Hydrogels by the Enzymatic Activity of the Incorporated Protease Substrate

Preparation of MMP-Sensitive Hydrogels with Various MMP Activity

Hydrogels were prepared as follows, with three different MMP-active oligopeptide substrate in the backbone: First, the adhesion peptide Ac-GCGYGRGDSPG-NH₂ was attached pendantly to a 4 arm-PEG-vinylsulfone (mol. wt. 15000) at a concentration of 0.1 mM by mixing the PEG precursor (TEOA buffer (0.3M, pH 8.0)) with the adhesion peptide also dissolved in the same buffer. The reaction was allowed to occur for 30 minutes at 37° C. Then, MMP-sensitive peptides of different activity (e.g. Ac-GCRDGPQGIWGQDRCG-NH₂) were mixed with the above solution still possessing Michael-type reactivity and gels were formed around a cell-fibrin clusters according to the method described in example 7. Samples were also cured in parallel and swelling was measured to guarantee that differences in cell migration could be plainly attributed to the change in enzymatic activity (and not differences in network architecture, i.e. X-link densities).

Cell Invasion Rate at a Given Adhesivity and Structure of the Network can be Rationally Tailored by the MMP Activity of the Incorporated Peptide Substrate

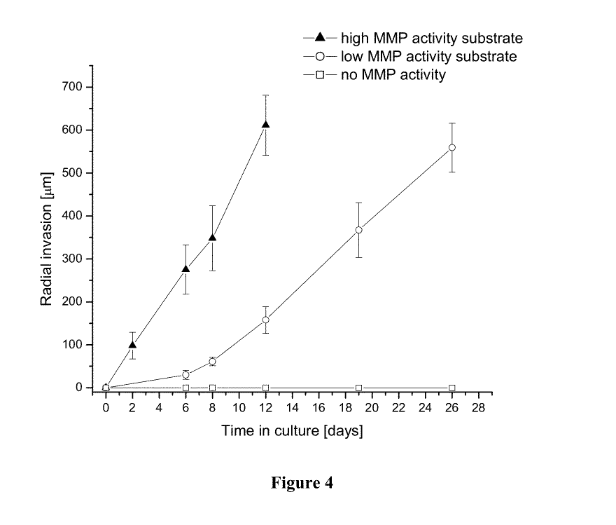

As expected from the biochemical measurements described in example 6, cellular invasion into hydrogels containing MMP-substrates responds to the enzymatic activity of the latter (FIG. 4). FIG. 4 is a graph of the amount of radial invasion as a function of incubation time for materials containing high MMP activity substrates, low MMP activity substrates, and MMP substrates having no activity. Thus, the kinetics of proteolytic gel breakdown can be engineered by very simple means. One synthetic substrate capable of forming a hydrogel by Michael-type addition was identified (GCRDGPQGIWGQDRCG) that degrades significantly faster than the peptide derived from a sequence found in the natural collagen type I (1α) chain (GCRDGPQGIAGQDRCG). Moreover, a peptide that is not sensitive to cell-secreted MMPs was identified.

### Example 9

### Changing Cell Invasion Rate into MMP-Sensitive Hydrogels by the Adhesion Site Density

Preparation of MMP-Sensitive Hydrogels with Various Adhesion Site Density

Hydrogels were prepared as follows, with various density of the adhesion peptide Ac-GCGYGRGDSPG-NH₂: First, adhesion peptides at a various concentrations were attached pendantly to a 4arm-PEG-vinylsulfone (mol. wt. 20000) by mixing the PEG precursor (TEOA buffer (0.3M, pH 8.0)) with the adhesion peptide also dissolved in the same buffer. The reaction was allowed to occur for 30 minutes at 37° C. Then, the MMP-sensitive peptide Ac-GCRDGPQGIWGQDRCG-NH₂ was mixed with the above solution still possessing Michael-type reactivity and gels were formed around a cell-fibrin clusters according to the method described in example 7. Samples were also cured in parallel and swelling was measured to guarantee that adhesion site density was constant in all gels after swelling and thus differences in cell migration could be plainly attributed to the change in network architecture.

Cell Invasion Rate at a Given MMP-Sensitivity and Network Architecture can be Rationally Tailored by the Adhesivity of the Network

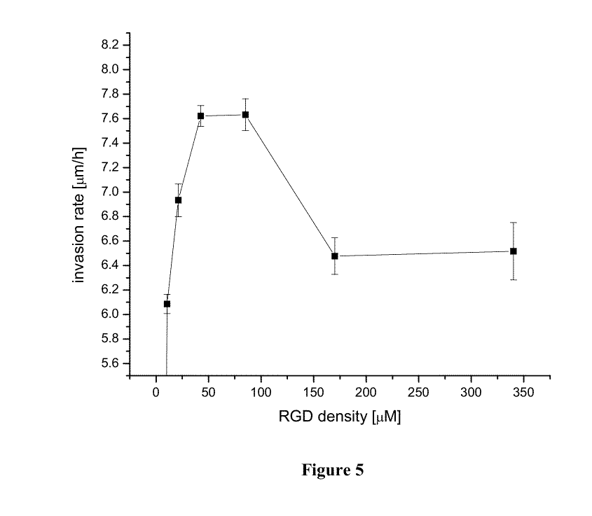

Three-dimensional cell invasion is mediated by the density of incorporated RGD sites (FIG. 5). FIG. 5 is a graph of invasion rate as a function of RGD density. HFF invasion rate depends in a biphasic manner on the concentration of adhesion ligands. We found a concentration regime that shows significantly higher migration rates than below or above the particular concentration. Thus, the kinetics of proteolytic gel breakdown can also be engineered by the adhesion site density.

### Example 10

### Changing Cell Infiltration Rate into MMP-Sensitive Hydrogels by the Molecular Weight (Structure and Number of Arms) of the Employed Macromer

Preparation of MMP-Sensitive Hydrogels with Various Network Architecture

Hydrogels were prepared as follows, with various PEG-VS macromers (4arm20 kD, 4arm15 kD, 4arm10 kD, 8arm20 kD): First, adhesion peptides at a given concentration of 0.1 mM (with regards to the swollen networks!) were attached pendantly to macromers by mixing the PEG precursor (TEOA buffer (0.3M, pH 8.0)) with the adhesion peptide also dissolved in the same buffer. The reaction was allowed to occur for 30 minutes at 37° C. Then, the MMP-sensitive peptide Ac-GCRDGPQGIWGQDRCG-NH₂ was mixed with the above solutions still possessing Michael-type reactivity and gels were formed around cell-fibrin clusters according to the method described in example 7. Samples were also cured in parallel and swelling was measured to guarantee that differences in cell migration could be plainly attributed to the change in adhesivity (and not differences in network architecture, i.e. X-link densities due to the various graft densities with pendant adhesion sites).

Cell Invasion Rate at a Given Adhesivity and MMP-Sensitivity of the Network can be Rationally Tailored by the MMP Activity of the Incorporated Peptide Substrate

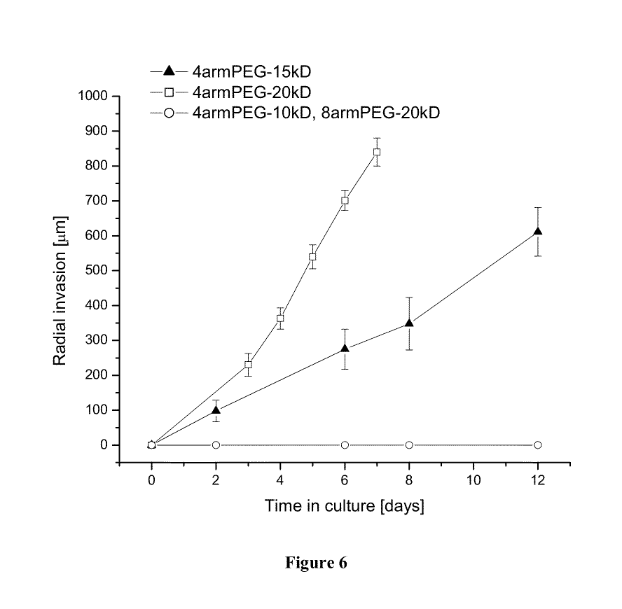

Cell invasion into synthetic gels is also mediated by the network architecture (FIG. 6). FIG. 6 is a graph showing that amount of radial invasion as a function of incubation time for materials having arms of different molecular weights and an increased degree of functionality. Cell invasion into synthetic gels increases with molecular weight. HFF invasion rate at constant RGD density and for the same MMP substrate increased with molecular weight. A threshold molecular weight (4armPEG10 kD) was found below which cell invasion essentially ceased. Thus, the kinetics of proteolytic gel breakdown can also be engineered by the network architecture.

### Example 11

### Increasing Cellular Infiltration by Loosening Up the Network Structure for Example Through Creation of Defects, and Switching Cell Migration from a Proteolytic to a Non-Proteolytic Mechanism

Preparation of MMP-Non-Sensitive and Adhesive Hydrogels that Allow Non-Proteolytic Cell Infiltration and Preparation of MMP-Sensitive and Adhesive Gels that Contain Large Amounts of Defects (Here: Dangling Ends)

Non-MMP sensitive hydrogels were prepared as follows: First, several known fraction of VS-group of a 4arm PEG-VS 20 kD macromer were reacted at 37° C. for 30 minutes with the amino acid cysteine to “kill” vinylsulfone functionalities prior to network formation in order to create networks with defects (i.e. pendant chains that would not contribute as elastically active chains). Then, the adhesion peptides at a given concentration of 0.1 mM (with regards to the swollen networks!) was attached pendantly to a 4arm PEG-VS 20 kD macromer by mixing the previously modified PEG precursor (TEOA buffer (0.3M, pH 8.0)) with the adhesion peptide also dissolved in the same buffer. The reaction was allowed to occur for 30 minutes at 37° C. Afterwards, this precursor was crosslinked with a PEG-dithiol (m.w. 3.4 kD). Swelling of samples were also conducted in parallel to control that differences in cell migration could be plainly attributed to the change in network architecture (i.e. creation of defect that loose up the network).

Similarly, MMP sensitive hydrogels were created with large amounts of defects by first reacting the PEG-VS macromers with the amino acid cysteine to “kill” vinylsulfone functionalities prior to network formation. Functionlization with adhesion sites and cross-linking was performed as described earlier.

Non Proteolytic Cell Invasion Occurs within Hydrogels with a Very Loosely X-Linked Network and Cellular Invasion can be Accelerated by Loosening Up the Network of MMP-Sensitive Gels

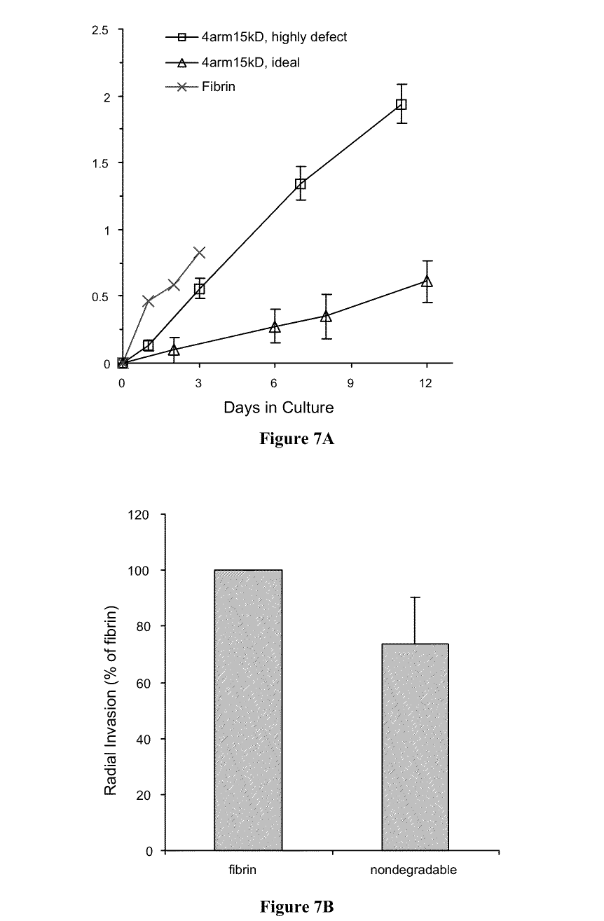

Networks can be created with non-MMP-sensitive molecules that still allow three-dimensional cell invasion to occur (FIG. 7B). FIG. 7B is a graph showing the amount of radial cell invasion (express as the % of radial invasion in fibrin) for a non-degradable matrix. However, a very high degree of defects, i.e. a very loosely X-linked network is necessary (G larger than ca. 10). Cell morphology is different from that found in proteolytically degradable matrices. Cells are very thin and spindle-shaped and migrate almost completely straight and radially out of the cluster. Thus, the mechanism of cellular infiltration can be switched from a predominantly proteolytic to a non-proteolytic one. By capping VS-groups with the amino acid Cys prior to cross-linking, MMP-sensitive gels with a very loosely X-linked architecture can be created. Cellular invasion of such matrices is significantly increased compared to the “perfect” networks (FIG. 7A). FIG. 7A is a graph of radial distance of cell invasion in mm as a function of incubation time for a highly defective matrix, an ideal matrix, and fibrin. Cell invasion rates for the highly defective matrix almost approached the rate of fibrin.

### Example 12

### Hydrogels of 4-Armed PEG-Itaconates 20K

Hydrogels were made with 4-armed PEG (MW 20K) functionalised by itaconates and bifunctional thiols, either in the form of peptides with cysteine residues, e.g. acetyl-GCRDGPQGIWGQDRCG-CONH or as thiol-PEG-thiol, e.g linear, MW 3.4K.

### Synthesis of 4-Armed PEG-Itaconates

### 4-hydrogen-1-methyl itaconate

102.1 g (0.65 mol) of dimethyl itaconate and 35.0 g (0.18 mol) of toluene-4-sulfonic acid monohydrate are dissolved in 50 ml of water and 250 ml of formic acid in a 1000 ml round bottom flask, equipped with a reflux condenser, a thermometer, and a magnetic stirring bar. The solution is brought to a light reflux by immersing the flask in an oil bath at 120° C. and is stirred for 45 min. Then, the reaction is quenched by pouring the slightly yellow, clear reaction mixture into 300 g of ice while stirring. The resulting clear aqueous solution is transferred to a separation funnel and the product is extracted by washing three times with 200 ml of dichloromethane. The combined organic layers are dried over MgSO₄ and the solvent is removed by rotary evaporation, yielding 64.5 g of raw product. Extracting the aqueous layer once more with 200 ml of dichloromethane yields another 6.4 g of raw product. A typical acidic smell indicates the presence of some formic acid in the fractions, which is removed by dissolving the combined fractions in 150 ml of dichloromethane and washing twice with 50 ml of saturated aqueous NaCl solution. Drying the organic layer with MgSO₄ and evaporating the solvent yields 60.1 g of a clear and colorless oil which is distilled under reduced pressure, yielding 55.3 g of a clear and colorless oil. According to ¹H NMR analysis the product consists for 91% of 4-hydrogen-1-methyl itaconate, for ca. 5% of 1-hydrogen-4-methyl itaconate, and for ca. 4% of dimethyl itaconate.

Gel Formation

Briefly, the precursor solutions were mixed 1:1 in stoichiometric balance of end groups. As was needed for reaction of thiols to vinyl sulfones and acrylates, the presence of triethanolamine in buffer form (TEOA) was required to promote the Michael reaction between thiols and itaconates.

The gel-forming rate of PEG-itaconates was dependent on the amount of base catalyst as well as on the resulting pH of the system. Table 1 presents the time (min) to onset of gelation for 10% w/w PEG-itaconate/PEG-thiol hydrogels with respect to TEOA buffer pH and concentration at room temperature (˜23° C.) and 37° C. (incubator/water bath*). Onset of gelation was defined as the point when the liquid precursor solution sticks to pipette tips used to probe the sample.

TABLE 1

| | Base/Buffer | pH | Onset of gelation, min |
|---|---|---|---|
| Room temperature | 0.15M TEOA | 10.2 | 6 |
| (23° C.) | | 9.5 | 10 |
| | | 9.1 | 17 |
| | | 8.6 | 25 |
| | | 8.4 | >40 |
| | 0.3M | 9.0 | 8 |
| | | 8.6 | 12.5 |
| | | 8.4 | 30.5 |
| 37° C. | 0.3M | >9.5 | 3.5 |
| | | 9.0 | <7.5/5 |
| | | 8.6 | 11/9 |
| | | 8.4 | 24/20 |
| | | 8.2 | 45/n.a. |
| | | 7.9 | 48/n.a. |

note:
gelation rates of samples in water bath were in general faster than those in incubator, likely due to better heat transfer for more actual temperature of reaction.

The itaconate-thiol reaction produced hydrogels with characteristics typical of 4-armed 20K PEG gels as formed through reaction of other functionalised end groups, e.g. VS or Ac. Physically, the gels were clear and soft, as previously described for PEG gels formed by reaction of other functionalised groups. In addition, 10% and 20% w/w gels swelled significantly after incubation in saline at 37° C. for 24 hours.

Cell Culture

PEG-itaconate/peptide hydrogels also supported in vitro cell culture in presence of added RGD peptides.

### Example 13

### Bone Regeneration

Bone Regeneration in the Rat Cranium

Animals were anesthetized by induction and maintenance with Halothan/O2. The surgical area was clipped and prepared with iodine for aseptic surgery. A linear incision was made from the nasal bone to the midsagital crest. The soft tissues were reflected and the periosteum was dissected from the site (occipital, frontal, and parietal bones). An eight mm craniotomy defect was created with a trephine in a dental hand piece, carefully avoiding dural perforation. The surgical area was flushed with saline to remove bone debris and a preformed gel was placed within the defect. The soft tissues were closed with skin staples. After the operation analgesia was provided by SQ injection of Buprenorphine (0.1 mg/kg). Rats were sacrificed by CO2 asphyxiation 21-35 days after implantation. Craniotomy sites with 5-mm contiguous bone were recovered from the skull and placed in 40% ethanol. At all steps, the surgeon was blinded regarding the treatment of the defects. Samples were sequentially dried: 40% ethanol (2 d), 70% ethanol (3 d), 96% ethanol (3 d), and 100% ethanol (3 d). Dried samples were defatted in xylene (3 d). Defatted samples were saturated (3 d) with methylmethacrylate (MMA, Fluka 64200) and then fixed at 4° C. by soaking (3 d) in MMA containing di-benzoylperoxide (20 mg/mL, Fluka 38581). Fixed samples were embedded in MMA, di-benzoylperoxide (30 mg/mL), and 100 μL/mL plastoid N or dibutylthalate (Merck) at 37° C. Sections (5 μm) were stained with Toluidine blue 0 and Goldner Trichrome. Histologic slides were scanned and the digital images processed with Leica QWin software.

Bone Healing in the Rat Cranium Defect Model can be Tailored by Several Matrix Characteristics

Synthetic hydrogels were used to induce de novo bone formation in vivo. Histological preparations indicated that the healing response largely depended on the composition of the hydrogel matrix. At a dose of 5 μg BMP-2 per implant MMP-sensitive peptides containing a fast degrading substrate, Ac-GCRDGPQGIWGQDRCG, and adhesive hydrogels were infiltrated by cells, predominantly fibroblast-like cells and intramembranous bone formation was observed. By 5 weeks, implant materials were fully resorbed, and new bone covered the defect area. Here, complete bridging of the defects was observed. Control materials made with a MMP-insensitive PEG-(SH)2 showed no cell infiltration and only bone formation around the intact gel implants. The slower degrading oligopeptide Ac-GCRDGPQGIAGQDRCG lead to significantly less cell infiltation. Thus, the healing response in vivo was dependent on the enzymatic activity of the incorporated substrate.

Gels with different structure were tested, including MMP-sensitive degradable gels made with 4armPEG-VS 15 kD, MMP-sensitive gels made with 4armPEG-VS-20 kD 20K and hydrolytically degradable gels made with PEG-dithiol 3.4 kD and 4armPEG-Acrylate, each at 12% w/w of the overall composition. In each animal complete bridging of the defects was observed at this early time point along with distinct morphology differences. The slower degrading gel showed less cell infiltration and more remaining matrix while the fastest degrading gel showed newly formed bone with morphology similar to original bone.

Bone Healing in the 8-Mm Sheep Drill Defect Model

8 mm drill defects were created in the tibia and femur of sheep and various synthetic matrices were polymerized in situ in the presence of 20 μg/mL of rhBMP-2 to test the ability of these matrices to induce healing of a boney defect. We proposed that it is crucial for a wound healing matrix to have strong cell infiltration characteristics, meaning cells can readily enter and remodel the synthetic matrix. As described earlier, we have shown in vitro and in other in vivo models that the details of the matrix, incorporating degradation sites, the composition of the matrix and the density of the matrix as examples, are crucial for functional cell infiltration. Within the development process outlined above, a series of materials with different cell infiltration characteristics were developed. Within this extensive series, five materials were tested in the sheep, representing a range of cell migration properties. These materials were labeled SRT 1-5, with SRT1 having the lowest cell infiltration characteristics. The amount of infiltration then increases through the series leading to SRT5 which allows the greatest amount of cell infiltration into the matrix. The animals were then allowed to heal for 8 weeks and were subsequently sacrificed and the defect region was excised for analysis via micro computerized topography (μCT) as well as histological analysis.

Bone Healing in the 8-Mm Sheep Drill Defect Model can be Tailored by Several Matrix Characteristics

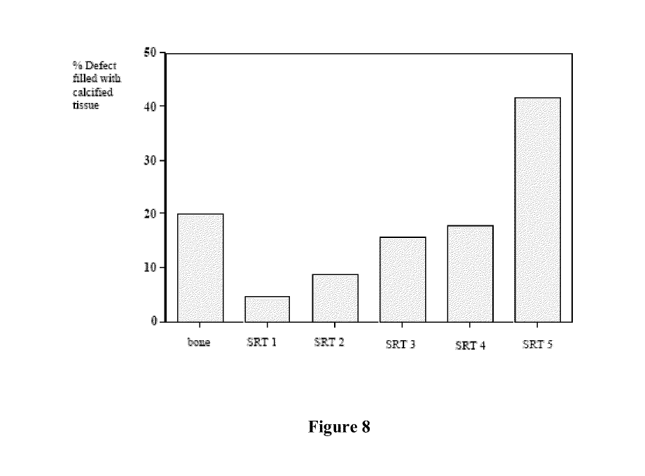

The five materials that were tested explored two different changes in the composition. SRT1 is a hydrogel with a plasmin degradation site incorporated into the backbone while SRT2 is a hydrogel with identical structure but with a collagenase degradation site in the backbone. These gels are made by mixing a peptide that each respective enzyme can cleave which is bracketed by two thiols (cysteines) which is then crosslinked with RGD modified 4arm15K PEG vinyl sulfone. The results are shown in FIG. 8. It can be seen that by changing the specificity of the enzyme that can degrade the gel, a different healing response is observed with the collagenase degradable sequence performing better. Additionally, the effect of structural aspects was explored as well. SRT2, SRT3 and SRT4 represent gels with decreasing crosslink density and it can be seen that the rate of healing is increased as the crosslink density decreases. SRT3 is made from a trithiol peptide and a linear PEG vinylsulfone while SRT4 is identical to SRT2 except that it has a 4arm20K PEG instead of a 4arm15K PEG, leading to lower crosslink density. This clearly will have a limit as a minimum crosslink density will be required to obtain gelation. Finally, SRT5 is a hydrolytically degradable matrix made from 4arm 15K PEG-acrylate and 3.4K PEG dithiol. These gels have the fastest degradation time and as such have the highest healing rate.

In analyzing these results, it is important to consider where the implants were located. These implants were placed within cancellous bone and as such, the entire volume of the bone is not filled with calcified tissue. When normal cancellous bone is analyzed via μCT, the bone volume fraction is approximately 20%. When μCT was employed to test the results of the various synthetic materials tested in the assay, newly formed calcified bone was found within the original defect. In some examples, the amount of bone was very substantial for the dose employed, leading to approximately 20% calcified volume as well. There was also a clear trend in the healing response with respect to the cell infiltration characteristics of the gels employed. Gels which gave limited ability for cells to infiltrate showed the lowest healing response, with newly formed calcified tissue only appearing at the margins of the defect and no calcified tissue at all in the center. In contrast, the materials that had faster cell infiltration properties showed a much higher healing response with a direct correlation between faster cell infiltration and better bone healing being observed.

These results were further confirmed by histology. When the histological sections were analyzed, it was observed that the boneless void in the center of “SRT1” actually represents gel that had not degraded at all. In each sample of the series, gel was observed, however materials with faster cell infiltration properties showed less remaining gel and more bone and precursor bone within the center of the defect. This clearly demonstrates that the bone was formed by infiltration of the surrounding cells into the matrix and subsequent conversion and formation of bone and bone matrix. In some examples, where the infiltration of cells into the matrix is slow, it is possible to block and inhibit regeneration. However, when a matrix is employed that has fast cell infiltration properties, then the amount of bone healing is dramatically enhanced leading to a excellent healing response.

Influence of Starting Concentration of First Precursor Molecule in the Healing Response in a Sheep Drill Hole Model

Two different starting concentrations of the enzymatic degradeable gels were employed. In each of these, the concentration of RGD and the active factor (Cp1PTH at 100 μg/mL) were kept constant. The polymeric network was formed from a four-arm branched PEG functionalized with four vinylsulfone endgroups of a molecular weight of 20 kD (molecular weight of each of the arms 5 kD) and dithiol peptide of the following sequence Gly-Cys-Arg-Asp-(Gly-Pro-Gln-Gly-Ile-Trp-Gly-Gln)-Asp-Arg-Cys-Gly. Both precursor molecules were dissolved in 0.3 M Triethanolamine. The starting concentration of the functionalized PEG (first precursor molecule) and the dithiol peptide (second precursor molecule) were varied. In one case the concentration was 12.6 weight % of the total weight of the composition. The second starting concentration was 9.5 weight % of the total weight of the composition. This has the consequence that the amount of dithiol peptide was changed such that the molar ratio between vinyl sulfones and thiols was maintained.

The gel which started from a starting concentration of 12.6 weight % swelled to a concentration of 8.9 weight % of total weight of the polymeric network plus water, thus the matrix had a water content of 91.1. The gel which started from a starting concentration of 9.5 weight % swelled to a final concentration of 7.4 weight % of total weight of the polymeric network plus water, thus had a water content of 92.6.

In order to explore the effect of this change, these materials were tested in the sheep drill defect. Here, a 750 μL defect was placed in the cancellous bone of the diaphyses of the sheep femur and humerus and filled with an in situ gelating enzymatic gel. The following amount of calcified tissue was obtained, determined via μCT, with each group at N=2:

| Starting concentration of gel | Calcified Tissue |
|---|---|
| 12.6% | 2.7% |
| 9.5% | 38.4% |

By making the gels less dense and easier for cell penetration, the resulting healing response with the addition of an active factor was stronger. The effect of having final solid concentrations of below 8.5 weight % is obvious from these results.

Clearly then, the design of the matrix is crucial to enable healing in wound defects. Each of these hydrogels were composed of large chains of polyethylene glycol, end linked to create a matrix. However, the details of how they were linked, via enzymatic degradation sites, the density of the linkers and several other variables were crucial to enable a functional healing response. These differences were very clearly observed in the sheep drill defect model.

### Example 14

### Hydrogels of 4-Arm PEG Acrylates

110 mg of pentaerythritol polyethyleneglycol ether tetraacrylate (4-arm PEG acrylate; MW=15 kD) was dissolved in 0.5 ml of TRIS/HCl buffer (pH 8.0) by vortexing for 10-15 seconds (Solution ACR). 45 mg of linear polyethyleneglycol dithiol was dissolved in 0.5 ml of TRIS/HCl buffer (pH 8.0) by vortexing for 10-15 seconds (Solution SH). 500 μL of Solution ACR was mixed with 500 μL of Solution SH in an Eppendorf tube and vortexed for 10 seconds.

The weight percent of the two precursor molecules, before crosslinking, was 13.74 weight % of the weight of the entire composition. The solution of the two precursor molecules had a gelation time from 3 to 4 min at pH 8.0 at a temperature between 18° C. and 30° C. The gelation time of a 27.5% PEG gel (total PEG content) in triethanol amine was on average 60% faster than the gelation rate of the 13.74% w/w PEG gel, whereas a 10.8% w/w PEG gel had a gelation rate, in triethanol amine, about 30% slower than that of the reference 13.74% w/w gel. The longer gelation time of the 10.8% w/w synthetic gel was shortened by increasing the pH of the triethanolamine buffer up to pH 8.5. This pH, however, is less bio-compatible than the one used in the current formulation. Further, the higher reactivity of the precursors at this pH is responsible for more defects in the resulting network, as the PEG molecules have no time to re-arrange in solution to ensure that each reactive group on the first precursor will react with its counterpart group on the second precursor. The presence of imperfections in the gel is reflected by a faster degradation time in vitro (degradation buffer: physiological phosphate buffer solution). Triethanol amine is also difficult to handle due to its sensitivity to light and air.

The 13.74% w/w PEG gels show gelation, handling and degradation properties suitable for clinical applications. When immersed in phosphate buffer at pH 7.4 during a degradation test (physiological phosphate buffer) the 13.74% w/w gel reaches equilibrium after 24 hours, with a weight increase of 300-400%.

1-34. (canceled)

35. A kit comprising:
i) a first molecule comprising a multifunctional polyethylene glycol comprising m electrophilic groups;
ii) a second molecule comprising a multifunctional polyethylene glycol comprising n nucleophilic groups; and
iii) a base solution,
wherein n+m is at least 5, and
wherein the sum of the weights of the first and second molecules is 8% to 16% by weight of the total weight of the combination of the first molecule, the second molecule, and the base solution.

36. The kit of claim 35, wherein the sum of the weights of the first and second molecules is 10% to 15% by weight of the total weight of the combination of the first molecule, the second molecule, and the base solution.

37. The kit of claim 35, wherein the sum of the weights of the first and second molecules is 12% to 14.5% by weight of the total weight of the combination of the first molecule, the second molecule, and the base solution.

38. The kit of claim 35, wherein the electrophilic and nucleophilic groups are located at the termini of the first and second molecules, respectively.

39. The kit of claim 35, wherein the electrophilic groups are conjugated unsaturated groups or conjugated unsaturated bonds selected from acrylates, vinyl sulfones, methacrylates, acrylamides, methacrylamides, acrylonitriles, vinylsulfones, 2- or 4-vinylpyridinium, maleimides, or quinones.

40. The kit of claim 35, wherein the electrophilic groups are selected from —CO₂N(COCH₂)₂, —CO₂H, —CHO, —CHOCH₂, —N═C═O, —N(COCH)₂, and —S—S—(C₅H₄N).

41. The kit of claim 35, wherein the nucleophilic groups are selected from amino-, thiol-, or hydroxyl-groups.

42. The kit of claim 35, wherein the first molecule is a polyethylene glycol comprising vinyl sulfone or acrylate groups and the second molecule is polyethylene glycol comprising thiol- or amine groups.

43. The kit of claim 35, wherein the base solution comprises sodium carbonate, sodium borate, or glycine.

44. A polymeric network, wherein the polymeric network is the reaction product of:
i) a first molecule comprising a multifunctional polyethylene glycol comprising m electrophilic groups;
ii) a second molecule comprising a multifunctional polyethylene glycol comprising n nucleophilic groups; and
iii) a base solution,
wherein n+m is at least 5, and
wherein the sum of the weights of the first and second molecules is 8% to 16% by weight of the total weight of the combination of the first molecule, the second molecule, and the base solution.

45. The polymeric network of claim 44, wherein the polymeric network is comprised in a hydrogel.

46. The polymeric network of claim 45, wherein the water content of the hydrogel is 85 to 96 weight % of the total weight of the polymeric network.

47. The polymeric network of claim 44, wherein the sum of the weights of the first and second molecules is 10% to 15% by weight of the total weight of the combination of the first molecule, the second molecule, and the base solution.

48. The polymeric network of claim 44, wherein the sum of the weights of the first and second molecules is 12% to 14.5% by weight of the total weight of the combination of the first molecule, the second molecule, and the base solution.

49. The polymeric network of claim 44, wherein the electrophilic and nucleophilic groups are located at the termini of the first and second molecules, respectively.

50. The polymeric network of claim 44, wherein the electrophilic groups are conjugated unsaturated groups or conjugated unsaturated bonds selected from acrylates, vinyl sulfones, methacrylates, acrylamides, methacrylamides, acrylonitriles, vinylsulfones, 2- or 4-vinylpyridinium, maleimides, or quinones.

51. The polymeric network of claim 44, wherein the electrophilic groups are selected from —CO₂N(COCH₂)₂, —CO₂H, —CHO, —CHOCH₂, —N═C═O, —N(COCH)₂, and —S—S—(C₅H₄N).

52. The polymeric network of claim 44, wherein the nucleophilic groups are selected from amino-, thiol-, or hydroxyl-groups.

53. The polymeric network of claim 44, wherein the first molecule is a polyethylene glycol comprising vinyl sulfone or acrylate groups and the second molecule is polyethylene glycol comprising thiol- or amine groups.

54. The polymeric network of claim 44, wherein the base solution comprises sodium carbonate, sodium borate, or glycine.
